SEQUENCE LISTING (6 sequences)

SEQ ID NO: 1 - protein, 282 aa, Homo sapiens [sequence omitted]

SEQ ID NO: 2 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 3 - protein, 475 aa, Homo sapiens [sequence omitted]

SEQ ID NO: 4 - protein, 105 aa, Homo sapiens
MRSFFSFLGEAFDGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 5 - protein, 130 aa, Homo sapiens
MSGRGKQGGKARAKAKTRSSRAGLQFPVGRVHRLLRKGNYAERVGAGAPVYLAAVLEYLTAEILELAGNAARDNKKTRIIPRHLQLAIRNDEELNKLLGKVTIAQGGVLPNIQAVLLPKKTESHHKAKGK

SEQ ID NO: 6 - protein, 24 aa, Artificial Sequence
  Synthetic oligonucleotide
TTAGGGTTAGGGTTAGGGTTAGGG

(19) United States
(12) Patent Application Publication
(10) Pub. No.: US 2020/0225222 A1
(43) Pub. Date: Jul. 16, 2020

(54) SLE DISEASE MANAGEMENT

(71) Applicants: IMMUNARRAY LTD., Tel Aviv (IL); YEDA RESEARCH AND DEVELOPMENT CO. LTD., Rehovot (IL)

(72) Inventors: Rachel SOREK, Moshav Tzafaria (IL); Keren JAKOBI-BROOK, Tel Aviv (IL); Pennina SAFER, Rehovot (IL); Irun R. COHEN, Rehovot (IL)

(21) Appl. No.: 16/650,894

(22) PCT Filed: Sep. 17, 2018
(86) PCT No.: PCT/IL2018/051039

§ 371 Date: Mar. 26, 2020

Related U.S. Application Data
(60) Provisional application No. 62/564,330, filed on Sep. 28, 2017.

(51) Int. Cl. G01N 33/564; G16B 20/20; G16B 25/30; G16B 40/20

(52) U.S. Cl. CPC G01N 33/564; G16B 20/20; G01N 2800/56; G16B 40/20; G01N 2800/104; G16B 25/30

(57) ABSTRACT

Assays, kits and methods useful in the field of systemic lupus erythematosus (SLE) diagnosis and management for determining and providing SLE treatment adjustment include methods for detecting SLE resolution and for adjusting treatment in a subject hitherto diagnosed as having SLE.

### FIELD OF THE INVENTION

The invention relates to the field of systemic lupus erythematosus (SLE) diagnosis and management, specifically to assays and methods for determining and providing SLE treatment adjustment.

### BACKGROUND OF THE INVENTION

Systemic lupus erythematosus (SLE) is a chronic systemic autoimmune disease that causes inflammation and injury in multiple organs, and leads to significant morbidity, mortality, and societal costs. Primarily a disease of women, SLE usually begins in young adulthood and can affect the skin, kidneys, joints, blood elements, and nervous system among other organs. SLE can be highly variable clinically, and is often characterized by recurrent episodes of flares and intensification of disease activity. Similar to most autoimmune diseases, the etiology of lupus is complex and likely involves both environmental and genetic factors.

SLE is associated with a large spectrum of autoantibodies. IgG antibodies to more than 100 different antigens including DNA, nucleosomes, histones, viral antigens, transcription factors and more have been reported in different SLE patients (Sherer et al., 2004, Semin. Arthritis. Rheum. 34:501-37). Surprisingly, there is no serologic diagnosis of SLE and SLE is diagnosed on the basis of eleven criteria defined by the American College of Rheumatology (ACR). These criteria include malar rash, discoid rash, photosensitivity, oral ulcers, arthritis, serositis, renal disorder, neurologic disorder, hematologic disorder (e.g., leucopenia, lymphopenia, hemolytic anemia or thrombocytopenia), immunologic disorder and antibody abnormalities (particularly anti-nuclear antibodies (ANA) and anti-DNA antibodies) (Tan et al., 1997, Arthritis Rheum 1997, 40:1725). According to these criteria, subjects can be clinically diagnosed with SLE if they meet at least four of the eleven criteria. Recently, the Systemic Lupus Collaborating Clinics (SLICC) revised these criteria, as reviewed in Petri et al. (Arthritis and Rheumatism, 2012, Vol. 64, pages 2677-2686). Nevertheless, SLE is still possible even in cases when less than four criteria are present.

Although the precise pathology of SLE is not clear, it is widely accepted that autoantibodies play an important role. Autoantibodies to DNA are highly heterogeneous with respect to their avidity, immunoglobulin subclass composition, cross-reactivity and complement fixing ability. A number of techniques have been utilized for DNA autoantibodies detection, including immunofluorescent assays (IFA), enzyme-linked immunosorbent assays (ELISAs) and radioimmunoassays (RIA). However, the clinical value of anti-dsDNA antibodies largely depends on the assay principle and analytical variables of the methods used to quantitate and immunologically characterize them. Because of its varied and variable manifestations, the diagnosis of SLE is difficult and problematic and may require several years of clinical referrals before a definitive diagnosis is made.

F. J. Quintana et al. (“Antigen-chip technology for accessing global information about the state of the body”, Lupus, 2006, Vol. 15(7), pages 428-30) describe the use of microarray technology and informatics to develop an antigen chip capable of detecting global patterns of antibodies binding to hundreds of antigens simultaneously. Lupus is disclosed to be one of the interests of the authors.

J. G. Hanly at al. (“Measurement of autoantibodies using multiplex methodology in patients with systemic lupus erythematosus”, Journal of Immunological Methods, 2010, Vol. 352, pages 147-152) have compared laser bead immunoassay technology to more traditional measures of autoantibody detection in diagnosis and assessment of SLE. The autoantigens used included, for example, dsDNA, Sm, and RNP.

Q. Z. Li et al. (“Protein array autoantibody profiles for insights into systemic lupus erythematosus and incomplete lupus syndromes”, Clinical & Experimental Immunology, 2006, Vol. 147 (1), pages 60-70) investigated the prevalence and clinical significance of a spectrum of autoantibodies in systemic lupus erythematosus and incomplete lupus syndromes using a proteome microarray bearing 70 autoantigens, such as ssDNA and U1 snRNP.

W. H. Robinson et al. (“Autoantigen microarrays for multiplex characterization of autoantibody responses”, Nature Medicine, 2002, Vol. 8, pages 295-301) describe and characterize arrays bearing 196 autoantigens containing the major autoantigens in eight distinct human autoimmune diseases, including systemic lupus erythematosus. The autoantigens included, for example, ssDNA, Sm/RNP and U1 snRNP.

International patent application publication no. WO 2011/099012 relates to methods and kits for diagnosing SLE in a subject. Particularly, WO 2011/099012 relates to a specific antibody profile useful in diagnosing SLE in a subject. International patent application publication no. WO 2014/091490 relates to methods and kits for diagnosing SLE or SSc in a subject. Particularly, WO 2014/091490 relates to a specific antibody reactivity profile useful in diagnosing SLE or scleroderma in a subject. International patent application publication no. WO 2015/101987 relates to method of assaying or monitoring the immunological competence of a subject. The method comprises measuring the levels of antibodies in a sample obtained from a subject to poly-guanine oligonucleotides. International patent application publication no. WO 2015/101988 relates to methods and kits for diagnosing SLE in a subject. Particularly, WO 2015/101988 relates to specific oligonucleotide antibody reactivities useful in diagnosing SLE in a subject. U.S. patent application publication no. 2017/0074875 relates to methods for identifying markers for SLE and to the markers identified with the aid of this method, which can differentiate between SLE and other autoimmune diseases and between different SLE subgroups. International patent application publication no. WO 2016/139659 relates to protein, peptide, polynucleotide and oligonucleotide antigens useful in diagnosing or monitoring an autoimmune disorder such as systemic lupus erythematosus (SLE) in a subject. The antigens listed in include inter alia at least four antigens selected from the group consisting of ssDNA, Sm, DNAse I, Histone III-S, Ro52, U1 snRNP, Collagen III, Apo-SAA, H2a and Oligo21.

Fattal and coworkers described the use of an antigen microarray and informatics analysis in investigating anti-DNA autoantibodies. Particularly, they examined IgM and IgG antibodies to poly-G and other oligonucleotides in the sera of healthy persons and those diagnosed with SLE, SSc, or pemphigus vulgaris (PV) (Immunology, 2015, Vol. 146(3):401-410). Putterman and coworkers described the development, verification and validation of a rule-out test for a definitive rule-out of a diagnosis of SLE. The test uses micro-array technology platform to identify discriminating patterns of circulating autoantibodies among SLE patients compared to self-declared healthy individuals (J. Immunol. Methods, 2016, Vol. 429:1-6).

Certain drugs and biological agents have been suggested as SLE therapies, and a number of them are currently indicated for treatment of the clinical signs of SLE. However, current treatments may be costly and insufficiently effective, and have potential risk of toxicity and adverse effects. While symptom relief, or disease remission, has been known to occur in SLE patients, only anecdotal evidence of apparently complete resolution of SLE have been reported, particularly in connection with drug-induced SLE.

### SUMMARY OF THE INVENTION

Applicants have recognized that developing a reliable test for detecting, evaluating and predicting whether SLE resolution may occur in a patient would be highly beneficial for determining treatment adjustment and disease management in SLE patients. More specifically, methods, assays and kits constructed according to the principles and embodiments of the invention detect SLE resolution and can adjust treatment in a subject hitherto diagnosed as having SLE.

The methods, assays and kits constructed according to the principles of the invention are based, in part, on the surprising discovery, that a shift in SLE disease status, regardless of changes in the manifestation of clinical disease symptoms, may be identified using a serological test determining autoantibody reactivities. Specifically, the invention is based in part, on findings obtained when using a microarray-based autoantibody test, in accordance with an embodiment of the invention, in the assessment of new patient populations. More specifically, the test, known to distinguish SLE patients from healthy subjects, was found to retain >90% sensitivity during the first 10 years of disease, irrespective of age at diagnosis or patient ethnicity; the stability of the exemplary SLE test signature was also found to be independent of SLE disease activity index (SLEDAI) score during this period. However, SLE test scores surprisingly demonstrated a decline towards autoimmune profiles more closely resembling those of healthy subjects starting about three years following diagnosis, wherein about 30% of the samples taken after 10 years of disease diagnosis were unexpectedly identified with negative SLE test results. In addition, a higher proportion of asymptomatic (SLEDAI=0) patients tested 10 years after initial diagnosis, were identified as having lower test scores and a shift to a non-SLE (SLE ruled-out) status.

Further, rather than the normalization of SLE signature being associated with drug-induced immune suppression, the opposite was unexpectedly discovered—an improvement in serological activity and decrease in corticosteroid use during the longitudinal follow-up, was found to be correlated with the reduction in SLE signature. These finding further demonstrate the applicability of SLE treatment adjustment to the new patient population, identified according to the inventive methods disclosed herein.

This patient population, newly identified by the inventive methods as described herein, represents a group of SLE patients that may be undergoing disease resolution, and patients amenable for long term treatment reduction or termination. Thus, the invention provides, in some embodiments, means for differentiating between patients appearing to be minimally symptomatic or asymptomatic due to drug-induced immune suppression or temporary disease remission, and patients manifesting stable disease resolution, persisting even in the absence of continuing clinical management or manipulation.

Thus, according to a first aspect of the invention, methods are provided for detecting resolution of systemic lupus erythematosus (SLE) in a subject having been diagnosed as having SLE. In another aspect of the invention, methods are provided for adjusting treatment in a subject having been diagnosed as having SLE. In yet another aspect of the invention, methods may be used for differentiating disease remission from disease resolution in a subject having been diagnosed as having SLE. As used herein, the phrase “having been diagnosed as having SLE” refers to a subject in which a clinical diagnosis of SLE has been determined typically at least three years earlier. More typically, the methods of the invention are particularly advantageous to evaluate subjects having been diagnosed as having SLE at least ten years earlier.

The methods of the invention may rely upon determination and comparison of reactivity patterns to a plurality of SLE-related antigens. Specifically, embodiments of the invention may include determination and comparison of reactivity patterns to a plurality of antigens selected from the group consisting of: Deoxyribonuclease I (DNAse I, single stranded DNA (ssDNA), Type III-S Histone (Histone IIIS), Type III collagen (Collagen III), Small Nuclear Ribonucleoprotein (U1 snRNP), 52 kDa Ro protein (Ro52), Smith antigen (Sm), Apo-SAA and Histone H2A (H2a), using advantageous supervised classification algorithms as detailed hereinbelow.

According to specific embodiments of the invention, the methods may include the steps of:

(i) providing a first sample obtained from the subject at a first time point and a second sample obtained from the same subject at a second, subsequent time point;

(ii) exposing antibodies in each of the two samples to at least four antigens selected from the group consisting of: ssDNA, Sm, DNAse I, Histone Ro52, U1 snRNP, Collagen III, Apo-SAA, H2a and Oligo21 to detect the respective reactivity patterns of said two samples to the at least four antigens;

(iii) calculating scores based on the reactivity patterns of said two samples by a supervised classification algorithm; and

(iv) comparing said scores obtained for said two samples.

In certain advantageous embodiments of the invention, the first time point precedes the second time point by at least ten years. In other embodiments, said subject has been diagnosed as having SLE at least three years earlier of the second time point. In other embodiments, said subject has been diagnosed as having SLE at least ten years earlier of the second time point. In other advantageous embodiments, said subject is asymptomatic at the second time point. In other embodiments, said subject has been diagnosed as having SLE at or before the first time point and is asymptomatic at the second time point. In other embodiments, said subject has been diagnosed as having SLE at or before the first time point and is asymptomatic at the second time point, and the first time point precedes the second time point by at least 3, preferably at least 4, 5, 6, 7, 8 or 9 years, most preferably by at least 10 years. Each possibility represents a separate embodiment of the invention. Thus, the methods of the invention may typically be employed on a subject that is asymptomatic at the second time point, wherein the first time point precedes the second time point by at least ten years and/or wherein said subject has been diagnosed as having SLE at least ten years earlier of said second time point. In another embodiment of the invention, the reactivity of antibodies includes IgG reactivities, IgM reactivities, or a combination thereof In another embodiment of the invention, the supervised classification algorithm is selected from the group consisting of support vector machines (SVMs), logistic regression (LR), quadratic discriminant analysis (QDA), and linear discriminant analysis (LDA). In another embodiment, the reactivity of antibodies includes IgG reactivities, IgM reactivities, or a combination thereof, and the supervised classification algorithm is selected from the group consisting of SVMs, LR, QDA, and LDA. According to advantageous embodiments, specific combinations of antigen reactivities and algorithms, further referred to herein as classifiers, are preferably employed.

In one embodiment of the invention, the reactivity pattern includes reactivities of IgG antibodies to ssDNA, Sm, DNAse I, Ro52 and U1 snRNP, and reactivities of IgM antibodies to Histone III-S, and the supervised classification algorithm is SVMs. The aforementioned classifier is herein referred to as the SVMs classifier.

In another embodiment of the invention the reactivity pattern includes reactivities of IgG antibodies to ssDNA, U1 snRNP, Ro52, Collagen III and Apo-SAA, and reactivities of IgM antibodies to Histone III-S, and the supervised classification algorithm is LR. The aforementioned classifier is herein referred to as the LR classifier.

In another embodiment of the invention the reactivity pattern includes reactivities of IgG antibodies to ssDNA, U1 snRNP, Sm, Apo-SAA and Ro52, and reactivities of IgM antibodies to H2a, and the supervised classification algorithm is QDA. The aforementioned classifier is herein referred to as the QDA classifier.

In another embodiment of the invention the reactivity pattern includes reactivities of IgG antibodies to ssDNA, U1 snRNP and Sm, and reactivities of IgM antibodies to Histone III-S, U1 snRNP and Oligo21, and the supervised classification algorithm is LDA. The aforementioned classifier is herein referred to as the LDA classifier.

According to embodiments of the invention, in which a lower score indicates an increased probability that said subject is not afflicted with SLE (i.e. the lower the score the greater is the probability that said subject is not afflicted with SLE), the method for detecting resolution of SLE further includes:

(v) determining that said subject has SLE resolution if there is a significant reduction of the score obtained for said second sample compared to the score obtained for said first sample.

It is to be understood, however, that the scores may also be determined such that a higher score indicates an increased probability that said subject is not afflicted with SLE (i.e. the higher the score, the greater is the probability that said subject is not afflicted with SLE). Accordingly, embodiments of the invention may alternatively and equivalently comprise embodiments in which a higher score indicates an increased probability that said subject is not afflicted with SLE, further comprising: (v) determining that said subject has SLE resolution if there is a significant enhancement of the score obtained for said second sample compared to the score obtained for said first sample.

According to additional embodiments of the invention, the methods may further include providing at least one additional sample at a time point preceding the second time point and anteceding the first time point (to be subjected to the same assay steps as the first and second sample). A consistent significant reduction along the time points may be used to determine SLE resolution and identify a subject as amenable for treatment adjustment according to these embodiments.

In a particular embodiment, said scores are calculated (e.g. by a supervised classification algorithm selected from the group consisting of LR, QDA, and LDA) in the range of 0 to 1, in which the lower the score the greater is the probability that said subject is not afflicted with SLE, and the significant reduction of said score obtained for said second sample compared to said score obtained for said first sample is of at least 0.1. In a particular embodiment, the LDA algorithm, or in another particular embodiment the LDA classifier, is used.

In one aspect, the invention provides a method for detecting resolution of SLE in a subject having been diagnosed as having SLE, the method comprising the steps of:

providing a first sample obtained from the subject at a first time point and a second sample obtained from the same subject at a second, subsequent time point, wherein said subject has been diagnosed as having SLE at least three years earlier of the second time point and is asymptomatic at said second time point;

(ii) exposing antibodies in each of the two samples to at least four antigens selected from the group consisting of: ssDNA, Sm, DNAse I, Histone III-S, Ro52, U1 snRNP, Collagen III, Apo-SAA, H2a and Oligo21 to detect the respective reactivity patterns of said two samples to the at least four antigens;

(iii) calculating scores based on the reactivity patterns of said two samples by a supervised classification algorithm, in which the lower the score the greater is the probability that said subject is not afflicted with SLE;

(iv) comparing said scores obtained for said two samples, and

(v) determining that said subject has SLE resolution if there is a significant reduction of the score obtained for said second sample compared to the score obtained for said first sample.

In one embodiment, the first time point precedes the second time point by at least ten years. Additionally or alternatively, said subject has been diagnosed as having SLE at least ten years earlier of said second time point. In another embodiment the reactivity of antibodies comprises IgG reactivities, IgM reactivities, or a combination thereof, and the supervised classification algorithm is selected from the group consisting of linear discriminant analysis (LDA), support vector machines (SVMs), logistic regression (LR), and quadratic discriminant analysis (QDA). In other embodiments, the reactivity pattern comprises reactivities of IgG antibodies to ssDNA, U1 snRNP and Sm, and reactivities of IgM antibodies to Histone III-S, U1 snRNP and Oligo21, and the supervised classification algorithm is LDA, or the reactivity pattern comprises reactivities of IgG antibodies to ssDNA, Sm, DNAse I, Ro52 and U1 snRNP, and reactivities of IgM antibodies to Histone III-S, and the supervised classification algorithm is SVMs, or the reactivity pattern comprises reactivities of IgG antibodies to ssDNA, U1 snRNP, Ro52, Collagen III and Apo-SAA, and reactivities of IgM antibodies to Histone III-S, and the supervised classification algorithm is LR, or the reactivity pattern comprises reactivities of IgG antibodies to ssDNA, U1 snRNP, Sm, Apo-SAA and Ro52, and reactivities of IgM antibodies to H2a, and the supervised classification algorithm is QDA.

In another embodiment the sample is selected from the group consisting of a serum sample, a plasma sample and a blood sample, and wherein the antigens are used in the form of an antigen probe set, an antigen array, or an antigen chip. In another embodiment said subject is undergoing SLE treatment selected from the group consisting of: nonsteroidal anti-inflammatory drugs (NSAIDs), corticosteroids, immunosuppressants, hydroxychloroquine, cyclophosphamide, immunomodulators, and TNF-α inhibitors.

In another embodiment said scores are calculated in the range of 0 to 1 in which the lower the score the greater is the probability that said subject is not afflicted with SLE, and the significant reduction of said score obtained for said second sample compared to said score obtained for said first sample is of at least 0.1.

Thus, in one exemplary embodiment, the method includes the steps of:

(i) providing a first sample obtained from the subject at a first time point and a second sample obtained from the same subject at a second, subsequent time point, wherein the first time point precedes the second time point by at least ten years, and said subject is asymptomatic at said second time point;

(ii) exposing antibodies in each of the two samples to a plurality of antigens selected from the group consisting of: ssDNA, Sm, DNAse I, Histone III-S, Ro52, U1 snRNP, Collagen III, Apo-SAA, H2a and Oligo21 to detect the respective reactivity patterns of said two samples to the plurality of antigens, and calculating scores based on the reactivity patterns of said two samples, in which the lower the score the greater is the probability that said subject is not afflicted with SLE, using a supervised classification algorithm, wherein:
a. the reactivity pattern includes reactivities of IgG antibodies to ssDNA, U1 snRNP, Ro52, Collagen III and Apo-SAA, and reactivities of IgM antibodies to Histone III-S, and the supervised classification algorithm is logistic regression (LR), or
b. the reactivity pattern includes reactivities of IgG antibodies to ssDNA, U1 snRNP, Sm, Apo-SAA and Ro52, and reactivities of IgM antibodies to H2a, and the supervised classification algorithm is quadratic discriminant analysis (QDA), or
c. the reactivity pattern includes reactivities of IgG antibodies to ssDNA, U1 snRNP and Sm, and reactivities of IgM antibodies to Histone III-S, U1 snRNP and Oligo21, and the supervised classification algorithm is linear discriminant analysis (LDA); or
d. the reactivity pattern includes reactivities of IgG antibodies to ssDNA, Sm, DNAse I, Ro52 and U1 snRNP, and reactivities of IgM antibodies to Histone III-S, and the supervised classification algorithm is support vector machines (SVMs);

(iii) comparing said scores obtained for said two samples, and determining that said subject has SLE resolution if there is a reduction of at least 0.1 in the score obtained for said second sample compared to the score obtained for said first sample.

In another embodiment, the method is used wherein:

a. the reactivity pattern consists of reactivities of IgG antibodies to ssDNA, U1 snRNP, Ro52, Collagen III and Apo-SAA, and reactivities of IgM antibodies to Histone III-S, and the supervised classification algorithm is LR, or

b. the reactivity pattern consists of reactivities of IgG antibodies to ssDNA, U1 snRNP, Sm, Apo-SAA and Ro52, and reactivities of IgM antibodies to H2a, and the supervised classification algorithm is QDA, or

c. the reactivity pattern consists of reactivities of IgG antibodies to ssDNA, U1 snRNP and Sm, and reactivities of IgM antibodies to Histone III-S, U1 snRNP and Oligo21, and the supervised classification algorithm is LDA; or

d. the reactivity pattern consists of reactivities of IgG antibodies to ssDNA, Sm, DNAse I, Ro52 and U1 snRNP, and reactivities of IgM antibodies to Histone III-S, and the supervised classification algorithm is SVMs.

In another embodiment, said scores are calculated in the range of 0 to 1 in which the lower the score the greater is the probability that said subject is not afflicted with SLE, and wherein said classification algorithm is selected from the group consisting of LR, QDA and LDA.

In another exemplary embodiment, the method includes the steps of:

(i) providing a first sample obtained from the subject at a first time point and a second sample obtained from the same subject at a second, subsequent time point, wherein said subject is asymptomatic at the second time point;

(ii) exposing antibodies in each of the two samples to a plurality of antigens selected from the group consisting of: ssDNA, Sm, DNAse I, Histone III-S, Ro52, U1 snRNP, Collagen III, Apo-SAA, H2a and Oligo21 to detect the respective reactivity patterns of said two samples to the plurality of antigens, and calculating scores based on the reactivity patterns of said two samples, in which the lower the score the greater is the probability that said subject is not afflicted with SLE, using a supervised classification algorithm, wherein:

a. the reactivity pattern includes reactivities of IgG antibodies to ssDNA, U1 snRNP, Ro52, Collagen III and Apo-SAA, and reactivities of IgM antibodies to Histone III-S, and the supervised classification algorithm is logistic regression (LR), or

b. the reactivity pattern includes reactivities of IgG antibodies to ssDNA, U1 snRNP, Sm, Apo-SAA and Ro52, and reactivities of IgM antibodies to H2a, and the supervised classification algorithm is quadratic discriminant analysis (QDA), or

c. the reactivity pattern includes reactivities of IgG antibodies to ssDNA, U1 snRNP and Sm, and reactivities of IgM antibodies to Histone III-S, U1 snRNP and Oligo21, and the supervised classification algorithm is linear discriminant analysis (LDA); or

d. the reactivity pattern includes reactivities of IgG antibodies to ssDNA, Sm, DNAse I, Ro52 and U1 snRNP, and reactivities of IgM antibodies to Histone III-S, and the supervised classification algorithm is support vector machines (SVMs);

(iii) comparing said scores obtained for said two samples; and

(iv) comparing said scores obtained for said two samples, and

(v) determining that said subject has SLE resolution if there is a reduction of at least 0.1 in the score obtained for said second sample compared to the score obtained for said first sample.

In another embodiment, said subject is undergoing SLE treatment, e.g. selected from the group consisting of: nonsteroidal anti-inflammatory drugs (NSAIDs), corticosteroids, immunosuppressants, hydroxychloroquine, cyclophosphamide, immunomodulators, and biological agents such as TNF-α inhibitors.

In another embodiment the method further includes reducing the dose and/or frequency of treatment or ceasing administration of treatment to said subject determined to have SLE resolution.

In another embodiment, the method may be used for adjusting treatment and further includes the step of:

(v) determining that said subject is amenable for treatment adjustment if there is a significant reduction of the score obtained for said second sample compared to the score obtained for said first sample.

Thus, in another aspect, there is provided a method for adjusting treatment in a subject having been diagnosed as having systemic lupus erythematosus (SLE) at least three years earlier, the method comprising the steps of:

(i) providing a first sample obtained from the subject at a first time point and a second sample obtained from the same subject at a second, subsequent time point;

(ii) exposing antibodies in each of the two samples to at least four antigens selected from the group consisting of: ssDNA, Sm, DNAse I, Histone III-S, Ro52, U1 snRNP, Collagen III, Apo-SAA, H2a and Oligo21 to detect the respective reactivity patterns of said two samples to the at least four antigens;

(iii) calculating scores based on the reactivity patterns of said two samples by a supervised classification algorithm, wherein the lower the score the greater is the probability that said subject is not afflicted with SLE;

(iv) comparing said scores obtained for said two samples, and

(v) determining that said subject is amenable for treatment adjustment if there is a significant reduction of the score obtained for said second sample compared to the score obtained for said first sample.

In another embodiment, the first time point precedes the second time point by at least ten years and/or said subject has been diagnosed as having SLE at least ten years earlier. In another embodiment said subject is asymptomatic at the second time point.

In another embodiment, the treatment adjustment includes reducing the dose and/or frequency of said treatment or ceasing administration of said treatment to said subject. In another embodiment said method further includes adjusting treatment in said subject determined to be amenable for treatment adjustment.

In another embodiment, said treatment is selected from the group consisting of: nonsteroidal anti-inflammatory drugs (NSAIDs), corticosteroids, immunosuppressants, hydroxychloroquine, cyclophosphamide, immunomodulators, and biological agents such as TNF-α inhibitors. For example, without limitation, the treatment may be e.g. NSAIDs, corticosteroids, myfortic, Methotrexate, Imuran, Abatacept, Hizentra, Gammagard, Octagam, Privigen, Arava, Plaquenil, Cyclophosphamide, Benlysta, Rituximab and Orenica.

In another embodiment said scores are calculated in the range of 0 to 1 in which the lower the score the greater is the probability that said subject is not afflicted with SLE, and the significant reduction of said score obtained for said second sample compared to said score obtained for said first sample is of at least 0.1.

In another exemplary embodiment, the method for adjusting treatment includes the steps of:

(i) providing a first sample obtained from the subject at a first time point and a second sample obtained from the same subject at a second, subsequent time point, wherein the first time point precedes the second time point by at least ten years;

(ii) exposing antibodies in each of the two samples to a plurality of antigens selected from the group consisting of: ssDNA, Sm, DNAse I, Histone III-S, Ro52, U1 snRNP, Collagen III, Apo-SAA, H2a and Oligo21 to detect the respective reactivity patterns of said two samples to the plurality of antigens, and calculating scores based on the reactivity patterns of said two samples, in which the lower the score the greater is the probability that said subject is not afflicted with SLE, using a supervised classification algorithm, wherein:

a. the reactivity pattern includes reactivities of IgG antibodies to ssDNA, U1 snRNP, Ro52, Collagen III and Apo-SAA, and reactivities of IgM antibodies to Histone III-S, and the supervised classification algorithm is logistic regression (LR), or

b. the reactivity pattern includes reactivities of IgG antibodies to ssDNA, U1 snRNP, Sm, Apo-SAA and Ro52, and reactivities of IgM antibodies to H2a, and the supervised classification algorithm is quadratic discriminant analysis (QDA), or

c. the reactivity pattern includes reactivities of IgG antibodies to ssDNA, U1 snRNP and Sm, and reactivities of IgM antibodies to Histone III-S, U1 snRNP and Oligo21, and the supervised classification algorithm is linear discriminant analysis (LDA); or

d. the reactivity pattern includes reactivities of IgG antibodies to ssDNA, Sm, DNAse I, Ro52 and U1 snRNP, and reactivities of IgM antibodies to Histone III-S, and the supervised classification algorithm is support vector machines (SVMs);

(iii) comparing said scores obtained for said two samples, and determining that said subject is amenable for treatment adjustment if there is a reduction of at least 0.1 in the score obtained for said second sample compared to the score obtained for said first sample.

In another exemplary embodiment, the method for adjusting treatment comprises the steps of:

(i) providing a first sample obtained from the subject at a first time point and a second sample obtained from the same subject at a second, subsequent time point, wherein the first time point precedes the second time point by at least ten years;

(ii) exposing antibodies in each of the two samples to a plurality of antigens selected from the group consisting of: ssDNA, Sm, DNAse I, Histone III-S, Ro52, U1 snRNP, Collagen III, Apo-SAA, H2a and Oligo21 to detect the respective reactivity patterns of said two samples to the plurality of antigens, and calculating scores based on the reactivity patterns of said two samples by a supervised classification algorithm, wherein:

a. the reactivity pattern includes reactivities of IgG antibodies to ssDNA, U1 snRNP, Ro52, Collagen III and Apo-SAA, and reactivities of IgM antibodies to Histone III-S, and the supervised classification algorithm is logistic regression (LR), or

b. the reactivity pattern includes reactivities of IgG antibodies to ssDNA, U1 snRNP, Sm, Apo-SAA and Ro52, and reactivities of IgM antibodies to H2a, and the supervised classification algorithm is quadratic discriminant analysis (QDA), or

c. the reactivity pattern includes reactivities of IgG antibodies to ssDNA, U1 snRNP and Sm, and reactivities of IgM antibodies to Histone III-S, U1 snRNP and Oligo21, and the supervised classification algorithm is linear discriminant analysis (LDA);

(iii) comparing said scores obtained for said two samples, and further comparing the score obtained for said second sample to a pre-determined threshold score, wherein said scores are calculated in the range of 0 to 1 and the pre-determined threshold score is 0.18; and

(iv) determining that said subject is amenable for treatment adjustment if there is a significant reduction of the score obtained for said second sample compared to the score obtained for said first sample, and if said score obtained for said second sample is within two standard deviations (SD) of said pre-determined threshold score.

In another embodiment of the invention, the sample is selected from the group consisting of a serum sample, a plasma sample and a blood sample. Additionally or alternatively, in the methods of the invention, the antigens are used in the form of an antigen probe set, an antigen array, or an antigen chip. In yet another embodiment of the invention, said treatment is selected from the group consisting of: NSAIDs, corticosteroids, myfortic, Methotrexate, Imuran, Abatacept, Hizentra, Gammagard, Octagam, Privigen, Arava, Plaquenil, Cyclophosphamide, Benlysta, Rituximab and Orenica.

In another embodiment the supervised classification algorithm is selected from the group consisting of support vector machines (SVMs), logistic regression (LR), quadratic discriminant analysis (QDA), and linear discriminant analysis (LDA), and the reactivity of antibodies comprises IgG reactivities, IgM reactivities, or a combination thereof. In another embodiment the reactivity pattern comprises reactivities of IgG antibodies to ssDNA, Sm, DNAse I, Ro52 and U1 snRNP, and reactivities of IgM antibodies to Histone III-S, and the supervised classification algorithm is SVMs.

In another embodiment the reactivity pattern comprises reactivities of IgG antibodies to ssDNA, U1 snRNP, Ro52, Collagen III and Apo-SAA, and reactivities of IgM antibodies to Histone III-S, and the supervised classification algorithm is LR, or the reactivity pattern comprises reactivities of IgG antibodies to ssDNA, U1 snRNP, Sm, Apo-SAA and Ro52, and reactivities of IgM antibodies to H2a, and the supervised classification algorithm is QDA, or the reactivity pattern comprises reactivities of IgG antibodies to ssDNA, U1 snRNP and Sm, and reactivities of IgM antibodies to Histone III-S, U1 snRNP and Oligo21, and the supervised classification algorithm is LDA.

In another embodiment of the invention, the reactivity pattern consists of reactivities of IgG antibodies to ssDNA, U1 snRNP, Ro52, Collagen III and Apo-SAA, and reactivities of IgM antibodies to Histone III-S, and the supervised classification algorithm is LR, or

the reactivity pattern consists of reactivities of IgG antibodies to ssDNA, U1 snRNP, Sm, Apo-SAA and Ro52, and reactivities of IgM antibodies to H2a, and the supervised classification algorithm is QDA, or

the reactivity pattern consists of reactivities of IgG antibodies to ssDNA, U1 snRNP and Sm, and reactivities of IgM antibodies to Histone III-S, U1 snRNP and Oligo21, and the supervised classification algorithm is LDA; or

the reactivity pattern consists of reactivities of IgG antibodies to ssDNA, Sm, DNAse I, Ro52 and U1 snRNP, and reactivities of IgM antibodies to Histone III-S, and the supervised classification algorithm is SVMs.

In another aspect of the invention, a kit includes: a) an antigen probe set, an antigen array, or an antigen chip including at least four antigens selected from the group consisting of: ssDNA, Sm, DNAse I, Histone III-S, Ro52, U1 snRNP, Collagen III, Apo-SAA, H2a and Oligo21; and b) instructions for use thereof for detecting SLE resolution in a subject having been diagnosed as having SLE. In another embodiment the kit may comprise a plurality of the antigens selected from the group consisting of ssDNA, Sm, DNAse I, Histone III-S, Ro52, U1 snRNP, Collagen III, Apo-SAA, H2a and Oligo21 such as a specific subset thereof as disclosed as being useful in the classifiers described herein. In another embodiment, the kit may further include means for detecting SLE resolution as disclosed herein.

In another aspect of the invention, a pharmaceutical pack includes: a) an SLE treatment, and b) instructions for treatment adjustment in a subject determined to be amenable for treatment adjustment as disclosed herein. In another embodiment the instructions may include reducing the dose and/or frequency of said treatment or ceasing administration of said treatment to said subject. In another embodiment, the SLE treatment may be e.g. nonsteroidal anti-inflammatory drugs (NSAIDs), corticosteroids, immunosuppressants, hydroxychloroquine, cyclophosphamide, immunomodulators, or biological agents such as TNF-α inhibitors. According to particular embodiments, said treatment may be e.g. NSAIDs, corticosteroids, myfortic, Methotrexate, Imuran, Abatacept, Hizentra, Gammagard, Octagam, Privigen, Arava, Plaquenil, Cyclophosphamide, Benlysta, Rituximab and Orenica. In another embodiment the pharmaceutical pack further contains an antigen probe set, an antigen array, or an antigen chip including at least four antigens (or a plurality of antigens) as disclosed herein and/or means for detecting SLE resolution as disclosed herein.

Other objects, features and advantages will become clear from the following description and drawings.

### BRIEF DESCRIPTION OF THE DRAWINGS

FIG. 1. Results of the SLE test on serum samples from patients obtained at three time points after diagnosis: up to 3 years; from 3 to 10 years; and greater than 10 years.

FIG. 2. The results of the SLE test over time in serum samples from clinically asymptomatic patients (SLEDAI=0) for the three time post diagnosis groups.

FIGS. 3A-3F. Reactivities of the individual markers among the three groups. FIG. 3A, ssDNA; FIG. 3B, U1 snRNP IgG; FIG. 3C, Histone III-S; FIG. 3D, U1 snRNP IgM; FIG. 3E, Sm; FIG. 3F, Oligo21.

FIG. 4. SLE Rule-Out score distribution of individual samples, grouped by the time after diagnosis, relative to healthy controls (HC).

FIG. 5. SLE Rule-Out score distribution of SLEDAI=0 patients, grouped by the time after diagnosis, relative to the HC.

### DETAILED DESCRIPTION OF THE INVENTION

The inventive concepts generally relate to the field of systemic lupus erythematosus (SLE) diagnosis and management, and, more specifically, to assays and methods for determining and providing SLE treatment adjustment. More specifically, embodiments of the invention relate to methods for detecting SLE resolution and for adjusting treatment in a subj ect hitherto diagnosed as having SLE.

The principles of the invention are based, in part, on the identification of a new patient population, which may be amenable for SLE treatment adjustment or termination. It is herein disclosed for the first time, that a fundamental change in disease state can occur in certain SLE patients, but only after years of established disease (typically more than three years and more typically after about ten years on average). This fundamental change may be detected according to the principles of the invention by monitoring dynamic changes in the lupus autoantibody signature. It is herein unexpectedly disclosed that long-term repeated SLE testing, to monitor these dynamic changes, can be useful in managing selected patients.

Hitherto reported serological tests attempted at identifying whether a subject is likely to be afflicted with lupus, and/or to monitor fluctuations in disease manifestation, which are characteristic of the typical course of disease (represented by either progressive deterioration or temporary attenuation of disease progression). These methods were typically employed by detecting certain biomarkers and comparing their levels to those of healthy control individuals. In contradistinction, the principles of the invention provide for differentiation between patients appearing to be minimally symptomatic or asymptomatic due to drug-induced immune suppression or temporary disease remission, and patients manifesting stable disease resolution. Thus, embodiments of the invention provide for shortening the course of treatment adjustment, and aiding the treating physician in determining appropriate therapeutic modalities with reduced trial and error, thereby minimizing both suffering for the patient and therapeutic costs alike.

In particular, it is surprisingly disclosed herein, that an immunoassay-based method may be applied to a pre-selected patient population with established SLE, at specific time intervals and using specific assay parameters, to monitor changes in the patient's immune signature to these antigens over time, thereby determining if the tested patient is undergoing

SLE resolution, and if the patient's treatment may be adjusted to minimize therapy-associated burden. The term resolution as used herein refers to a stable and persisting alleviation of the disease, even in the absence of continuing clinical management or manipulation. Thus, this term as used herein is distinguishable from the apparent short-term reduction in disease manifestation, which may be associated with drug-induced immune suppression or temporary disease remission. Without wishing to be bound by a specific theory or mechanism of action, SLE resolution may be characterized by attenuation of autoimmune processes underlying the etiology and/or pathology of the disease. Accordingly, the group of patients under SLE resolution, newly identified herein, are typically characterized by normalization of SLE serological activity and/or relief of SLE symptoms, often appearing as asymptomatic subjects. Thus, these subjects may appear as having low disease activity scores (e.g. SLEDAI=0). However, it is to be understood, that as serological changes often precede changes in clinical manifestations in lupus, the methods of the invention may be used for early detection of SLE resolution, even in patients that are still minimally symptomatic. As demonstrated herein, these patients may be further characterized by apparently normalized anti-double stranded (ds) DNA antibodies, serum C3, and serum C4.

In one aspect, the invention relates to a method for detecting resolution of systemic lupus erythematosus (SLE) in a subject having been diagnosed as having SLE, the method including the steps of:

(i) providing a first sample obtained from the subject at a first time point and a second sample obtained from the same subject at a second, subsequent time point, wherein said subject is asymptomatic at the second time point;

(ii) exposing antibodies in each of the two samples to at least four antigens selected from the group consisting of: ssDNA, Sm, DNAse I, Histone III-S, Ro52, U1 snRNP, Collagen III, Apo-SAA, H2a and Oligo21 to detect the respective reactivity patterns of said two samples to the at least four antigens;

(iii) calculating scores based on the reactivity patterns of said two samples by a supervised classification algorithm, wherein the lower the score the greater is the probability that said subject is not afflicted with SLE;

(iv) comparing said scores obtained for said two samples, and

(v) determining that said subject has SLE resolution if there is a significant reduction of the score obtained for said second sample compared to the score obtained for said first sample.

In one embodiment, said subject has been diagnosed as having SLE at least three years earlier of the second time point and is asymptomatic at said second time point. In another embodiment the first time point precedes the second time point by at least ten years and/or said subject has been diagnosed as having SLE at least ten years earlier of said second time point. In another embodiment, the first time point precedes the second time point by at least ten years. In another embodiment said subject has been diagnosed as having SLE at least ten years earlier of said second time point. In another embodiment the reactivity of antibodies includes IgG reactivities, IgM reactivities, or a combination thereof, and wherein the supervised classification algorithm is selected from the group consisting of linear discriminant analysis (LDA), support vector machines (SVMs), logistic regression (LR), and quadratic discriminant analysis (QDA). In another embodiment the reactivity pattern includes reactivities of IgG antibodies to ssDNA, U1 snRNP and Sm, and reactivities of IgM antibodies to Histone III-S, U1 snRNP and Oligo21, and the supervised classification algorithm is LDA. In another embodiment the reactivity pattern includes reactivities of IgG antibodies to ssDNA, Sm, DNAse I, Ro52 and U1 snRNP, and reactivities of IgM antibodies to Histone III-S, and the supervised classification algorithm is SVMs. In another embodiment the reactivity pattern includes reactivities of IgG antibodies to ssDNA, U1 snRNP, Ro52, Collagen III and Apo-SAA, and reactivities of IgM antibodies to Histone III-S, and the supervised classification algorithm is LR. In another embodiment the reactivity pattern includes reactivities of IgG antibodies to ssDNA, U1 snRNP, Sm, Apo-SAA and Ro52, and reactivities of IgM antibodies to H2a, and the supervised classification algorithm is QDA. In another embodiment the sample is selected from the group consisting of a serum sample, a plasma sample and a blood sample, and wherein the antigens are used in the form of an antigen probe set, an antigen array, or an antigen chip. In another embodiment said subject is undergoing SLE treatment selected from the group consisting of: nonsteroidal anti-inflammatory drugs (NSAIDs), corticosteroids, immunosuppressants, hydroxychloroquine, cyclophosphamide, immunomodulators, and TNF-α inhibitors. In another embodiment said scores are calculated in the range of 0 to 1 in which the lower the score the greater is the probability that said subject is not afflicted with SLE, and the significant reduction of said score obtained for said second sample compared to said score obtained for said first sample is of at least 0.1.

In another embodiment, the method includes the steps of:

(i) providing a first sample obtained from the subject at a first time point and a second sample obtained from the same subject at a second, subsequent time point, wherein the first time point precedes the second time point by at least ten years, and said subject is asymptomatic at said second time point;

(ii) exposing antibodies in each of the two samples to a plurality of antigens selected from the group consisting of: ssDNA, Sm, DNAse I, Histone III-S, Ro52, U1 snRNP, Collagen III, Apo-SAA, H2a and Oligo21 to detect the respective reactivity patterns of said two samples to the plurality of antigens, and calculating scores, based on the reactivity patterns of said two samples, in which the lower the score the greater is the probability that said subject is not afflicted with SLE, using a supervised classification algorithm, wherein:

a. the reactivity pattern includes reactivities of IgG antibodies to ssDNA, U1 snRNP, Ro52, Collagen III and Apo-SAA, and reactivities of IgM antibodies to Histone III-S, and the supervised classification algorithm is logistic regression (LR), or

b. the reactivity pattern includes reactivities of IgG antibodies to ssDNA, U1 snRNP, Sm, Apo-SAA and Ro52, and reactivities of IgM antibodies to H2a, and the supervised classification algorithm is quadratic discriminant analysis (QDA), or

c. the reactivity pattern includes reactivities of IgG antibodies to ssDNA, U1 snRNP and Sm, and reactivities of IgM antibodies to Histone III-S, U1 snRNP and Oligo21, and the supervised classification algorithm is linear discriminant analysis (LDA); or

d. the reactivity pattern includes reactivities of IgG antibodies to ssDNA, Sm, DNAse I, Ro52 and U1 snRNP, and reactivities of IgM antibodies to Histone III-S, and the supervised classification algorithm is support vector machines (SVMs);

(iii) comparing said scores obtained for said two samples, and determining that said subject has SLE resolution if there is a reduction of at least 0.1 in the score obtained for said second sample compared to the score obtained for said first sample.

In another embodiment of the method:

a. the reactivity pattern consists of reactivities of IgG antibodies to ssDNA, U1 snRNP, Ro52, Collagen III and Apo-SAA, and reactivities of IgM antibodies to Histone III-S, and the supervised classification algorithm is LR, or

b. the reactivity pattern consists of reactivities of IgG antibodies to ssDNA, U1 snRNP, Sm, Apo-SAA and Ro52, and reactivities of IgM antibodies to H2a, and the supervised classification algorithm is QDA, or

c. the reactivity pattern consists of reactivities of IgG antibodies to ssDNA, U1 snRNP and Sm, and reactivities of IgM antibodies to Histone III-S, U1 snRNP and Oligo21, and the supervised classification algorithm is LDA; or

d. the reactivity pattern consists of reactivities of IgG antibodies to ssDNA, Sm, DNAse I, Ro52 and U1 snRNP, and reactivities of IgM antibodies to Histone III-S, and the supervised classification algorithm is SVMs.

In another embodiment said scores are calculated in the range of 0 to 1 in which the lower the score the greater is the probability that said subject is not afflicted with SLE, and wherein said classification algorithm is selected from the group consisting of LR, QDA and LDA.

In another embodiment said method further includes reducing the dose and/or frequency of treatment or ceasing administration of treatment to said subject determined to have SLE resolution.

In another aspect, there is provided a method for adjusting treatment in a subject having been diagnosed as having systemic lupus erythematosus (SLE) at least three years earlier, the method comprising the steps of:

(i) providing a first sample obtained from the subject at a first time point and a second sample obtained from the same subject at a second, subsequent time point;

(ii) exposing antibodies in each of the two samples to at least four antigens selected from the group consisting of: ssDNA, Sm, DNAse I, Histone III-S, Ro52, U1 snRNP, Collagen III, Apo-SAA, H2a and Oligo21 to detect the respective reactivity patterns of said two samples to the at least four antigens;

(iii) calculating scores based on the reactivity patterns of said two samples by a supervised classification algorithm, wherein the lower the score the greater is the probability that said subject is not afflicted with SLE;

(iv) comparing said scores obtained for said two samples, and

(v) determining that said subject is amenable for treatment adjustment if there is a significant reduction of the score obtained for said second sample compared to the score obtained for said first sample.

In another aspect, there is provided a method for adjusting treatment in a subject having been diagnosed as having systemic lupus erythematosus (SLE) at least ten years earlier, the method comprising the steps of:

(i) providing a first sample obtained from the subject at a first time point and a second sample obtained from the same subject at a second, subsequent time point;

(ii) exposing antibodies in each of the two samples to at least four antigens selected from the group consisting of: ssDNA, Sm, DNAse I, Histone III-S, Ro52, U1 snRNP, Collagen III, Apo-SAA, H2a and Oligo21 to detect the respective reactivity patterns of said two samples to the at least four antigens;

(iii) calculating scores based on the reactivity patterns of said two samples by a supervised classification algorithm, wherein the lower the score the greater is the probability that said subject is not afflicted with SLE;

(iv) comparing said scores obtained for said two samples, and

(v) determining that said subject is amenable for treatment adjustment if there is a significant reduction of the score obtained for said second sample compared to the score obtained for said first sample.

In another embodiment the treatment adjustment includes reducing the dose and/or frequency of said treatment or ceasing administration of said treatment to said subject. In another embodiment the method further includes adjusting treatment in said subject determined to be amenable for treatment adjustment. In another embodiment the first time point precedes the second time point by at least ten years. In another embodiment said subject is asymptomatic at the second time point. In another embodiment the supervised classification algorithm is selected from the group consisting of support vector machines (SVMs), logistic regression (LR), quadratic discriminant analysis (QDA), and linear discriminant analysis (LDA), and the reactivity of antibodies includes IgG reactivities, IgM reactivities, or a combination thereof In another embodiment the reactivity pattern includes reactivities of IgG antibodies to ssDNA, Sm, DNAse I, Ro52 and U1 snRNP, and reactivities of IgM antibodies to Histone III-S, and the supervised classification algorithm is SVMs. In another embodiment the reactivity pattern includes reactivities of IgG antibodies to ssDNA, U1 snRNP, Ro52, Collagen III and Apo-SAA, and reactivities of IgM antibodies to Histone III-S, and the supervised classification algorithm is LR, In another embodiment the reactivity pattern includes reactivities of IgG antibodies to ssDNA, U1 snRNP, Sm, Apo-SAA and Ro52, and reactivities of IgM antibodies to H2a, and the supervised classification algorithm is QDA. In another embodiment the reactivity pattern includes reactivities of IgG antibodies to ssDNA, U1 snRNP and Sm, and reactivities of IgM antibodies to Histone III-S, U1 snRNP and Oigo21, and the supervised classification algorithm is LDA. In another embodiment the sample is selected from the group consisting of a serum sample, a plasma sample and a blood sample, and wherein the antigens are used in the form of an antigen probe set, an antigen array, or an antigen chip. In another embodiment said treatment is selected from the group consisting of: nonsteroidal anti-inflammatory drugs (NSAIDs), corticosteroids, immunosuppressants, hydroxychloroquine, cyclophosphamide, immunomodulators, and TNF-α inhibitors. In another embodiment said treatment is selected from the group consisting of: NSAIDs, corticosteroids, myfortic, Methotrexate, Imuran,

Abatacept, Hizentra, Gammagard, Octagam, Privigen, Arava, Plaquenil, Cyclophosphamide, Benlysta, Rituximab and Orenica. In another embodiment said scores are calculated in the range of 0 to 1 in which the lower the score the greater is the probability that said subject is not afflicted with SLE, and the significant reduction of said score obtained for said second sample compared to said score obtained for said first sample is of at least 0.1.

In another embodiment, the method includes the steps of:

(i) providing a first sample obtained from the subject at a first time point and a second sample obtained from the same subject at a second, subsequent time point, wherein the first time point precedes the second time point by at least ten years;

(ii) exposing antibodies in each of the two samples to a plurality of antigens selected from the group consisting of: ssDNA, Sm, DNAse I, Histone III-S, Ro52, U1 snRNP, Collagen III, Apo-SAA, H2a and Oligo21 to detect the respective reactivity patterns of said two samples to the plurality of antigens, and calculating scores based on the reactivity patterns of said two samples, in which the lower the score the greater is the probability that said subject is not afflicted with SLE, using a supervised classification algorithm, wherein:

a. the reactivity pattern includes reactivities of IgG antibodies to ssDNA, U1 snRNP, Ro52, Collagen III and Apo-SAA, and reactivities of IgM antibodies to Histone III-S, and the supervised classification algorithm is logistic regression (LR), or

b. the reactivity pattern includes reactivities of IgG antibodies to ssDNA, U1 snRNP, Sm, Apo-SAA and Ro52, and reactivities of IgM antibodies to H2a, and the supervised classification algorithm is quadratic discriminant analysis (QDA), or

c. the reactivity pattern includes reactivities of IgG antibodies to ssDNA, U1 snRNP and Sm, and reactivities of IgM antibodies to Histone III-S, U1 snRNP and Oligo21, and the supervised classification algorithm is linear discriminant analysis (LDA); or

d. the reactivity pattern includes reactivities of IgG antibodies to ssDNA, Sm, DNAse I, Ro52 and U1 snRNP, and reactivities of IgM antibodies to Histone III-S, and the supervised classification algorithm is support vector machines (SVMs);

(iii) comparing said scores obtained for said two samples, and determining that said subject is amenable for treatment adjustment if there is a reduction of at least 0.1 in the score obtained for said second sample compared to the score obtained for said first sample.

In another embodiment, the method includes the steps of:

(i) providing a first sample obtained from the subject at a first time point and a second sample obtained from the same subject at a second, subsequent time point, wherein the first time point precedes the second time point by at least ten years;

(ii) exposing antibodies in each of the two samples to a plurality of antigens selected from the group consisting of: ssDNA, Sm, DNAse I, Histone III-S, Ro52, U1 snRNP, Collagen III, Apo-SAA, H2a and Oligo21 to detect the respective reactivity patterns of said two samples to the plurality of antigens, and calculating scores based on the reactivity patterns of said two samples by a supervised classification algorithm, wherein:

a. the reactivity pattern includes reactivities of IgG antibodies to ssDNA, U1 snRNP, Ro52, Collagen III and Apo-SAA, and reactivities of IgM antibodies to Histone III-S, and the supervised classification algorithm is logistic regression (LR), or

b. the reactivity pattern includes reactivities of IgG antibodies to ssDNA, U1 snRNP, Sm, Apo-SAA and Ro52, and reactivities of IgM antibodies to H2a, and the supervised classification algorithm is quadratic discriminant analysis (QDA), or

c. the reactivity pattern includes reactivities of IgG antibodies to ssDNA, U1 snRNP and Sm, and reactivities of IgM antibodies to Histone III-S, U1 snRNP and Oligo21, and the supervised classification algorithm is linear discriminant analysis (LDA);

(iii) comparing said scores obtained for said two samples, and further comparing the score obtained for said second sample to a pre-determined threshold score, wherein said scores are calculated in the range of 0 to 1 and the pre-determined threshold score is 0.18; and

(iv) determining that said subject is amenable for treatment adjustment if there is a significant reduction of the score obtained for said second sample compared to the score obtained for said first sample, and if said score obtained for said second sample is within two standard deviations (SD) of said pre-determined threshold score.

In another embodiment the reactivity pattern consists of reactivities of IgG antibodies to ssDNA, U1 snRNP, Ro52, Collagen III and Apo-SAA, and reactivities of IgM antibodies to Histone III-S, and the supervised classification algorithm is LR, In another embodiment the reactivity pattern consists of reactivities of IgG antibodies to ssDNA, U1 snRNP, Sm, Apo-SAA and Ro52, and reactivities of IgM antibodies to H2a, and the supervised classification algorithm is QDA, In another embodiment the reactivity pattern consists of reactivities of IgG antibodies to ssDNA, U1 snRNP and Sm, and reactivities of IgM antibodies to Histone III-S, U1 snRNP and Oligo21, and the supervised classification algorithm is LDA. In another embodiment the reactivity pattern consists of reactivities of IgG antibodies to ssDNA, Sm, DNAse I, Ro52 and U1 snRNP, and reactivities of IgM antibodies to Histone III-S, and the supervised classification algorithm is SVMs.

In another embodiment said treatment is selected from the group consisting of: NSAIDs, corticosteroids, immunosuppressants, hydroxychloroquine, cyclophosphamide, immunomodulators, and TNF-α inhibitors and said method includes reducing the dose and/or frequency of said treatment or ceasing administration of said treatment to said subject.

In another aspect, a kit includes:

a) an antigen probe set, an antigen array, or an antigen chip including at least four antigens selected from the group consisting of: ssDNA, Sm, DNAse I, Histone III-S, Ro52, U1 snRNP, Collagen III, Apo-SAA, H2a and Oligo21; and

b) instructions for use thereof for detecting SLE resolution in a subject having been diagnosed as having SLE.

Antigens

As defined herein, “U1 SnRNP” refers to a ribonuclear protein, which is conserved between species. In a particular embodiment, Small Nuclear Ribonucleoprotein 70 kDa Human Recombinant (U1 SnRNP) is commercially available, e.g., from Prospec, catalog number pro-445.

“DNAse I” is considered the major serum nuclease. DNAse I is the founding member of the DNAse I-like family of divalent cation-dependent endonucleases. In a particular embodiment, DNAse I antigen is commercially available, e.g., from AKRON biotech, catalog number AK3778.

“Histones” are the chief protein components of chromatin. They act as spools around which DNA winds and they play a role in gene regulation. Six major histone classes are known: H1 (sometimes called the linker histone; also related to Histone H5); H2A; H2B; H3; H4; and archaeal histones. Two each of the class H2A, H2B, H3 and H4, so-called core histones, assemble to form one octameric nucleosome core particle by wrapping 146 base pairs of DNA around the protein spool in 1.65 left-handed super-helical turn. The linker histone H1 binds the nucleosome and the entry and exit sites of the DNA, thus locking the DNA into place and allowing the formation of higher order structure. The most basic such formation is the 10 nm fiber or beads on a string conformation. This involves the wrapping of DNA around nucleosomes with approximately 50 base pairs of DNA spaced between each nucleosome (also referred to as linker DNA). The assembled histones and DNA is called chromatin. Higher order structures include the 30 nm fiber (forming an irregular zigzag) and 100 nm fiber, these being the structures found in normal cells. During mitosis and meiosis, the condensed chromosomes are assembled through interactions between nucleosomes and other regulatory proteins. In a particular embodiment, Histone H2A human antigen is commercially available, e.g., from Sigma Aldrich, catalog number H9250. In another particular embodiment, Histone Type III-S calf antigen is commercially available, e.g., from Sigma Aldrich, catalog number H5505.

Single Strand Deoxyribonucleic Acid (ssDNA)

The reactivity of antibodies to the ssDNA antigen may be determined according to techniques known in the art. The ssDNA antigen may be obtained from any source, such as but not limit to, calf, human, horse, pig or bovine source. In a particular embodiment, ssDNA has a CAS number of 91080-16-9. The ssDNA antigen is commercially available, e.g., from Sigma Aldrich, catalog number D8899.

Collagen Type III

Type III collagen is the second most abundant collagen in human tissues and occurs particularly in tissues exhibiting elastic properties, such as skin, blood vessels and various internal organs. Mutations of type III collagen cause the most severe form of Ehlers-Danlos syndrome, EDS IV, which affect arteries, internal organs, joints and skin, and may cause sudden death when the large arteries rupture. In a particular embodiment, the type III collagen antigen of the present invention is a Bornstein and Traub Type III collagen, e.g., from human placenta. The reactivity of antibodies to the collagen-III antigen may be determined according to techniques known in the art. In a particular embodiment, collagen-III has a CAS number of 9007-34-5. The collagen-III antigen is commercially available, e.g., from Sigma Aldrich, catalog number C4407.

Ro52

The function of the Ro52 protein has not been fully established, although a role in ubiquitination and other regulatory processes has been proposed. Ro52 includes several predicted functional domains; two zinc-finger motifs are situated in the N-terminal region and a SPRY-region is near the C-terminus. The central part of Ro52 consists of a coiled-coil region, including a leucine zipper comprising amino acid (aa) residues 200-232. Leucine zippers, which contain periodic repeats of leucine amino acids every seventh residue, give rise to a helical structure, and are likely to be of importance for the correct folding of the protein, as well as its interaction with other molecules. Based on an analysis of sequence similarity, the 475 amino acid (aa) protein Ro52 belongs to the tripartite motif (TRIM) family. In a particular embodiment, the Ro52 antigen is commercially available, e.g., from Prospec catalog number PRO-328.

Sm

Sm antigen is a non-histone nuclear protein composed of several polypeptides of differing molecular weights. They include B (26 kD), B′ (27 kD), and D (13 kD). The principle reactivity has been shown to reside in the B, B′, and D polypeptides. The Sm antigen is involved in normal post-transcriptional, premessenger RNA processing to excise introns. It has been demonstrated that the Sm antigenicity is both RNase and DNase resistant and partially resistant to tryptic digestion. In a particular embodiment, the Sm antigen is commercially available, e.g., from US Biological catalog number s1014-29F.

Apo-SAA

Human Apo-SAA is a 104 amino acid polypeptide that circulates primarily in association with high-density lipoproteins (HDL). The level of Apo-SAA, normally 1-5 μg/ml in plasma, increases 500-1000 fold within 24 hours of an inflammatory stimulus and, under these conditions, is the most abundant HDL apolipoprotein. The human SAA gene codes for a 122 amino acid polypeptide, which contains an 18 amino acid N-terminal signal sequence. Recombinant Apo-SAA is a consensus SAA molecule corresponding to human Apo-SAA1α, except for the presence of an N-terminal methionine, the substitution of asparagine for aspartic acid at position 60, and arginine for histidine at position 71 (the latter two substituted residues are present in Apo-SAA2β). The calculated molecular weight of Recombinant Human Apo-SAA is 11.7 kDa. In a particular embodiment, the Apo-SAA antigen is commercially available, e.g., from Peprotec catalog number 300-13.

Samples and Subject Population

In certain embodiments of the invention, the methods are preceded by a step including obtaining or deriving a sample from the subject. In certain embodiments, the sample is obtained or derived from the subject by non-invasive means or methods.

According to additional embodiments of the invention, the sample obtained from the subject is a biological fluid. According to some embodiments, the sample is selected from the group consisting of plasma, serum, blood, cerebrospinal fluid, synovial fluid, sputum, urine, saliva, tears, lymph specimen, or any other biological fluid known in the art. Each possibility represents a separate embodiment of the invention. According to certain embodiments, the sample obtained from the subject is selected from the group consisting of serum, plasma and blood. According to one embodiment, the sample is a serum sample. In certain embodiments, the sample is obtained or derived from the subject by non-invasive means or methods.

According to embodiments of the invention, the methods and assays as disclosed herein are used to evaluate subjects having been diagnosed as having SLE, i.e. subjects previously determined as being afflicted with SLE according to methods or criteria accepted in the art, e.g. according to ACR criteria or SLICC criteria as detailed below.

The 1982 American College of Rheumatology (ACR) criteria describe features necessary to diagnose SLE. The presence of as few as 4 of the 11 criteria yields a sensitivity of 85% and a specificity of 95% for SLE. Patients with SLE may present with any combination of clinical features and serologic evidence of lupus. The ACR's criteria are (1) Serositis (pleurisy, pericarditis on examination or diagnostic ECG or imaging), (2) Oral ulcers (oral or nasopharyngeal, usually painless; palate is most specific), (3) Arthritis (nonerosive, two or more peripheral joints with tenderness or swelling), (4) Photosensitivity (unusual skin reaction to light exposure), (5) Blood disorders (leukopenia (<4×10″ cells\hat{ }L on more than one occasion), lymphopenia (<1500 cells\hat{ }L on more than one occasion), thrombocytopenia (<100×10 cells\hat{ }L in the absence of offending medications), hemolytic anemia), (6) Renal involvement (proteinuria (>0.5 g/d or 3+ positive on dipstick testing) or cellular casts), (7) ANAs (higher titers generally more specific (>1: 160); must be in the absence of medications associated with drug-induced lupus), (8) Immunologic phenomena (dsDNA; anti-Smith (Sm) antibodies; antiphospholipid antibodies (anticardiolipin immunoglobulin G [IgG] or immunoglobulin M [IgM] or lupus anticoagulant); biologic false-positive serologic test results for syphilis, lupus erythematosus (LE) cells (omitted in 1997)), (9) Neurologic disorder (seizures or psychosis in the absence of other causes), (10) Malar rash (fixed erythema over the cheeks and nasal bridge, flat or raised), and (11) Discoid rash (erythematous raised-rimmed lesions with keratotic scaling and follicular plugging, often scarring).

The Systemic Lupus Collaborating Clinics (SLICC) recently revised and validated the American College of Rheumatology (ACR) SLE classification criteria in order to improve clinical relevance, meet stringent methodology requirements and incorporate new knowledge in SLE immunology (Petri et al., Arthritis and Rheumatism, 2012, Vol. 64, pages 2677-2686). Seventeen criteria were identified, including 11 clinical criteria and 6 immunological criteria. The SLICC criteria for SLE classification requires fulfillment of at least four criteria, with at least one clinical criterion and one immunologic criterion, or lupus nephritis as the sole clinical criterion in the presence of ANA or anti-dsDNA antibodies.

Two of the most commonly used instruments for evaluation of SLE patients are the Systemic Lupus Erythematosus Disease Activity Index (SLEDAI) and the Systemic Lupus Activity Measure (SLAM). The SLEDAI is an index that measures disease activity by weighting the importance of each organ system involved. The SLEDAI includes 24 items, representing nine organ systems. The variables are obtained by history, physical examination and laboratory assessment. Each item is weighted from 1 to 8 based on the significance of the organ involved. For example, mouth ulcers are scored as 2, while seizures are scored as 8. The laboratory parameters that are included in the SLEDAI include white blood cell count, platelet count, urinalysis, serum C3, C4 and anti-dsDNA. The total maximum score is 105.

Asymptomatic patients are characterized as SLEDAI=0.

The SLAM includes 32 items representing 11 organ systems. The items are scored not only as present/absent, but graded on a scale of 1 to 3 based on severity. Both the SLEDAI and the SLAM have been shown to be valid, reliable, and sensitive to change over time, and are widely used in research protocols and clinical trials. These indices are particularly useful for examining the value of newly proposed serologic or inflammatory markers of disease activity in SLE. Despite the obvious utility of these instruments, there are some drawbacks. First, there is not always complete agreement between the SLAM and the SLEDAI in the same set of patients. There are several possible reasons for these discrepancies. Unlike the SLEDAI, the SLAM includes constitutional symptoms such as fatigue and fever, which may or may not be considered attributable to active SLE; this activity index relies on physician interpretation. In addition, the SLEDAI does not capture mild degrees of activity in some organ systems and does not have descriptors for several types of activity, such as hemolytic anemia.

According to the principles of the invention, the methods and assays of the invention are used to evaluate established SLE patients known to have the disease for years, typically for three years or more, preferably at least 4, 5, 6, 7, 8 or 9 years, and more typically at least ten years. Thus, the methods of the invention are typically performed on subjects having been diagnosed as having SLE at least ten years earlier (of the second time point for sample collection as described herein) and/or wherein the time interval between the collection of the test samples is of at least ten years (such that the first time point precedes the second time point by at least ten years). Further, the subject evaluated for SLE resolution by the methods of the invention is typically asymptomatic at the second time point of sample collection.

As demonstrated herein, in subjects belonging to this newly defined population, SLE resolution may occur, which can be identified using the methods and assays as disclosed herein.

Accordingly, methods of the invention contain a step of sample collection, comprising providing a first sample obtained from a subject as defined herein at a first time point and a second sample obtained from the same subject at a second, subsequent time point, at time intervals as described herein.

Antibodies and Immunoassays

According to the principles of the invention, exposing antibodies in each of the two samples to the antigens is performed under conditions such that a specific antigen-antibody complex may be formed and subsequently detected as disclosed herein. Detection and relative quantification of the formed antigen-antibody complexes enables the detection of the respective reactivity patterns.

Thus, according to exemplary embodiments, step (ii) of exposing antibodies in each of the two samples to the at least four antigens, to detect the respective reactivity patterns of said two samples to said at least four antigens, is performed by a process comprising:

a. contacting each sample, under conditions such that a specific antigen-antibody complex may be formed, with an antigen probe set comprising said plurality of antigens (e.g. at least four antigens as disclosed herein), and

b. quantifying the amount of antigen-antibody complex formed for each antigen probe, to detect the respective reactivity patterns of each sample to said antigens.

The amount of antigen-antibody complex is indicative of the level of the tested antibody in the sample (or the reactivity of the sample with the antigen). It is to be understood that this step needs not be performed simultaneously; for example, the first sample may be exposed to the antigens at the first time point and the second sample may be exposed to the antigens at the second time point, to obtain their respective reactivity patterns, wherein the corresponding scores calculated may be subsequently compared.

As used herein, the “reactivity of antibodies in a sample” to a plurality of antigens (e.g. at least four antigens) refers to the immune reactivity of each antibody in the sample to a specific antigen selected from the plurality of antigens. The immune reactivity of the antibody to the antigen, i.e. its ability to specifically bind the antigen, may be used to determine the amount of the antibody in the sample. The calculated levels of each one of the tested antibodies in the sample are selectively referred to as the reactivity pattern of the sample to these antigens.

The term “plurality of antigens” as used herein refers to at least four antigens. Thus, these terms may be used interchangeably according to embodiments of the invention as described herein. According to still further embodiments, the reactivity of antibodies is determined to a plurality of antigens such as to at least 5 antigens, alternatively to at least 6 antigens, 7 antigens, 8 antigens, or 9 antigens. According to yet further embodiments, the plurality of antigens used in the methods and kits of the invention may comprise or consist of the entire set of 10 antigens, namely ssDNA, Sm, DNAse I, Histone III-S, Ro52, U1 snRNP, Collagen III, Apo-SAA, H2a and Oligo21. In other embodiments, the plurality of antigens used in the methods and kits of the invention may comprise or consist of the subsets as disclosed herein, namely ssDNA, U1 snRNP, Sm, Histone III-S, and Oligo21, alternatively ssDNA, Sm, DNAse I, Ro52, Histone III-S and U1 snRNP, alternatively ssDNA, U1 snRNP, Ro52, Collagen III, Histone III-S and Apo-SAA, alternatively ssDNA, U1 snRNP, Sm, Apo-SAA, H2a and Ro52. Each possibility represents a separate embodiment of the invention.

An antibody “directed to” an antigen, as used herein is an antibody which is capable of specifically binding the antigen.

It should be understood that when the terms “antibody” or “antibodies” are used, this is intended to include intact antibodies, such as polyclonal antibodies or monoclonal antibodies (mAbs), as well as proteolytic fragments thereof such as the Fab or F(ab′)₂ fragments. Further included within the scope of the invention (for example as immunoassay reagents, as detailed herein) are chimeric antibodies; recombinant and engineered antibodies, and fragments thereof.

The term “antigen” as used herein is a molecule or a portion of a molecule capable of being bound by an antibody. The antigen is typically capable of inducing an animal to produce antibody capable of binding to an epitope of that antigen. An antigen may have one or more epitopes. The specific reaction referred to above is meant to indicate that the antigen will react, in a highly selective manner, with its corresponding antibody and not with the multitude of other antibodies which may be evoked by other antigens. An “antigenic oligonucleotide” is an oligonucleotide which is capable of specifically binding an antibody.

In another embodiment, detection of the capacity of an antibody to specifically bind an antigen probe may be performed by quantifying specific antigen-antibody complex formation. The term “specifically bind” as used herein means that the binding of an antibody to an antigen probe is not competitively inhibited by the presence of non-related molecules.

In accordance with the principles of the invention, any suitable immunoassay can be used with the subject antigens. Such techniques are well known to the ordinarily skilled artisan and have been described in many standard immunology manuals and texts. In certain preferable embodiments, determining the capacity of the antibodies to specifically bind the antigen probes is performed using an antigen probe array-based method. Preferably, the array is incubated with suitably diluted serum of the subject (e.g. diluted 1:10) so as to allow specific binding between antibodies contained in the serum and the immobilized antigen probes, washing out unbound serum from the array, incubating the washed array with a detectable label-conjugated ligand of antibodies of the desired isotype, washing out unbound label from the array, and measuring levels of the label bound to each antigen probe.

In other embodiments, various immunoassays may be used, including, without limitation, enzyme-linked immunosorbent assay (ELISA), flow cytometry with multiplex beads (such as the system made by Luminex), surface plasmon resonance (SPR), elipsometry, and various other immunoassays which employ, for example, laser scanning, light detecting, photon detecting via a photo-multiplier, photographing with a digital camera based system or video system, radiation counting, fluorescence detecting, electronic, magnetic detecting and any other system that allows quantitative measurement of antigen-antibody binding.

Various methods have been developed for preparing arrays suitable for the methods of the invention. State-of-the-art methods involves using a robotic apparatus to apply or “spot” distinct solutions containing antigen probes to closely spaced specific addressable locations on the surface of a planar support, typically a glass support, such as a microscope slide, which is subsequently processed by suitable thermal and/or chemical treatment to attach antigen probes to the surface of the support. Suitable supports may also include silicon, nitrocellulose, paper, cellulosic supports and the like.

Preferably, each antigen probe, or distinct subset of antigen probes that may be used in embodiments of the invention, which is attached to a specific addressable location of the array, is attached independently to at least two, more preferably to at least three separate specific addressable locations of the array in order to enable generation of statistically robust data.

In addition to antigen probes, the array may advantageously include control antigen probes or other standard chemicals. Such control antigen probes may include normalization control probes. The signals obtained from the normalization control probes provide a control for variations in binding conditions, label intensity, “reading” efficiency and other factors that may cause the signal of a given binding antibody-probe ligand interaction to vary. For example, signals, such as fluorescence intensity, read from all other antigen probes of the antigen probe array are divided or subtracted or shifted according to difference in mean intensities by the signal (e.g., fluorescence intensity) from the normalization control probes thereby normalizing the measurements. Normalization control probes can be bound to various addressable locations on the antigen probe array to control for spatial variation in antibody-ligand probe efficiency. Normalization control probes can be located at the corners or edges of the array to control for edge effects, as well as in the middle of the array.

The labeled antibody ligands may be of any of various suitable types of antibody ligand. Preferably, the antibody ligand is an antibody which is capable of specifically binding the Fc portion of the antibodies of the subject used. For example, where the antibodies of the subject are of the IgG isotype, the antibody ligand is preferably an antibody capable of specifically binding to the Fc region of IgG antibodies of the subject.

The ligand of the antibodies of the subject may be conjugated to any of various types of detectable labels. Preferably the label is a fluorophore, most preferably Cy3. Alternately, the fluorophore may be any of various fluorophores, including Cy5, fluorescein isothiocyanate (FITC), phycoerythrin (PE), rhodamine, Texas red, and the like. Suitable fluorophore-conjugated antibodies specific for antibodies of a specific isotype are widely available from commercial suppliers and methods of their production are well established.

Antibodies of the subject may be isolated for analysis of their antigen probe binding capacity in any of various ways, depending on the application and purpose. While the subject's antibodies may be suitably and conveniently in the form of blood serum or plasma or a dilution thereof (e.g. 1:10 dilution), the antibodies may be subjected to any desired degree of purification prior to being tested for their capacity to specifically bind antigen probes. The method of the invention may be practiced using whole antibodies of the subject, or antibody fragments of the subject which include an antibody variable region.

In certain other exemplary embodiments, detecting said reactivity patterns may comprise:

(I) bioinformatically clustering said antibodies and said antigens and identifying all stable antibody and antigen clusters;

(II) scanning said antigen clusters, while using reactivity levels of antigens of each antigen cluster as a feature set representing first object sets containing either all of said antibodies or any of said stable antibody clusters;

(III) scanning said antibody clusters, while using reactivity levels of antibody of each antibody cluster as a feature set representing second object sets containing either all of said antigens or any of said stable antigen clusters;

(IV) tracking all antibody and antigen stable clusters thus generated;

(V) repeating steps (I)-(IV) until no new antibody and antigen stable clusters being generated, thereby obtaining final stable antigens and antibody clusters and pointers identifying how all of said stable antibody and antigen clusters have been generated.

Data Analysis

According to the principles of the invention, the methods disclosed herein employ the use of supervised classification algorithms, e.g. learning and pattern recognition algorithms as disclosed herein, for calculating scores based on the antibody reactivity patterns detected for the two samples collected at the predetermined time interval. The scores, also referred to herein as SLE scores, SLE probability scores or probability scores, represent the likelihood of the tested subject to be afflicted or not afflicted with SLE. The scores are then compared to determine whether a significant score change occurs over that time interval.

Supervised classifiers are prediction tools based on learning from examples of labeled data. A supervised classification algorithm is a form of learning and pattern recognition algorithm, in which labeled data, consisting of input (typically vector)-output (correct classification) pairs, is used to train the classifier. Through the training process, a classification function is inferred from labeled training data. The classification function can then be used for classifying new examples, thereby correctly determining the class labels for unseen instances.

In certain embodiments, the learning and pattern recognition algorithm is SVM. In machine learning, support vector machines (SVMs, also support vector networks) are supervised learning models with associated learning algorithms that analyze data and recognize patterns, used for classification and regression analysis. Given a set of training examples, each marked as belonging to one of two categories, an SVM training algorithm builds a model that assigns new examples into one category or the other, making it a non-probabilistic binary linear classifier. An SVM model is a representation of the examples as points in space, mapped so that the examples of the separate categories are divided by a clear gap that is as wide as possible. New examples are then mapped into that same space and predicted to belong to a category based on which side of the gap they fall on.

In certain embodiments, the learning and pattern recognition algorithm is logistic regression (LR). In statistics, logistic regression, or logit regression, or logit model is a type of probabilistic statistical classification model. It is also used to predict a binary response from a binary predictor, used for predicting the outcome of a categorical dependent variable (i.e., a class label) based on one or more predictor variables (features). That is, it is used in estimating the parameters of a qualitative response model. The probabilities describing the possible outcomes of a single trial are modeled, as a function of the explanatory (predictor) variables, using a logistic function. Frequently “logistic regression” is used to refer specifically to the problem in which the dependent variable is binary, that is, the number of available categories is two.

“Logistic regression” is part of a category of statistical models called generalized linear models. Logistic regression allows one to predict a discrete outcome, such as group membership, from a set of variables that may be continuous, discrete, dichotomous, or a mix of any of these. The dependent or response variable is dichotomous, for example, one of two possible types of cancer. Logistic regression models the natural log of the odds ratio, i.e., the ratio of the probability of belonging to the first group (P) over the probability of belonging to the second group (1-P), as a linear combination of the different expression levels (in log-space) and of other explaining variables. The logistic regression output can be used as a classifier by prescribing that a case or sample will be classified into the first type if P is greater than 0.5 or 50%. Alternatively, the calculated probability P can be used as a variable in other contexts such as a 1D or 2D threshold classifier.

In certain embodiments, the learning and pattern recognition algorithm is linear discriminant analysis (LDA). LDA and the related Fisher's linear discriminant are methods used in statistics, pattern recognition and machine learning to find a linear combination of features which characterizes or separates two or more classes of objects or events. The resulting combination may be used as a linear classifier or, more commonly, for dimensionality reduction before later classification.

In certain embodiments, the learning and pattern recognition algorithm is Quadratic Discriminant analysis (QDA). A quadratic classifier is used in machine learning and statistical classification to separate measurements of two or more classes of objects or events by a quadric surface. It is a more general version of the linear classifier. QDA is closely related to LDA, where it is assumed that the measurements from each class are normally distributed. Unlike LDA however, in QDA there is no assumption that the covariance of each of the classes is identical. When the normality assumption is true, the best possible test for the hypothesis that a given measurement is from a given class is the likelihood ratio test.

In certain embodiments, the learning and pattern recognition algorithm is Classification and Decision Tree (CART). Decision tree learning uses a decision tree as a predictive model which maps observations about an item to conclusions about the item's target value. It is one of the predictive modelling approaches used in statistics, data mining and machine learning. Tree models where the target variable can take a finite set of values are called classification trees. In these tree structures, leaves represent class labels and branches represent conjunctions of features that lead to those class labels.

The score represents the predicted probability of a given patient to not be an SLE patient, given two alternatives: SLE and healthy control. Accordingly, the score calculated according to embodiments of the invention is presented in the range of 0 to 1. The prediction is performed by an appropriate supervised classification algorithm as disclosed herein, trained and validated on a data set of SLE patients and healthy controls, using a plurality of antigens as defined herein as its input. For example, prediction may be performed by an LDA classifier using a 6-feature antigen intensity vector as its input, trained and validated on a data set of SLE patients and healthy controls.

In another embodiment a change of at least 0.1 in the score obtained for said second sample compared to the score obtained for said first sample, wherein said scores are calculated in the range of 0 to 1, is considered a significant score change in the methods of the invention. Thus, in cases in which said scores are calculated in the range of 0 to 1 wherein the lower the score the greater is the probability that said subject is not afflicted with SLE, a reduction of at least 0.1 is considered a significant score reduction, indicative of SLE resolution and/or amenability for treatment adjustment.

In other exemplary embodiments, said scores are further compared to a pre-determined threshold score, wherein said scores are calculated in the range of 0 to 1 and the pre-determined threshold score is 0.18, and wherein a significant reduction of the score obtained for said second sample compared to the score obtained for said first sample, and further wherein said score obtained for said second sample is within two standard deviations (SD) of said pre-determined threshold score, is considered a significant score change in the methods of the invention.

Treatment and Treatment Adjustment

According to additional embodiments, the principles of the invention provide for adjusting treatment in a subject having been diagnosed as having SLE. More specifically, a subj ect identified as undergoing SLE resolution and/or as manifesting a significant score change over time by the methods disclosed herein, may be amenable for adjustment of their treatment schedule, so as to reduce the incidence and/or severity of treatment-associated side effects and adverse events without enhancing the incidence and/or severity of disease associated symptoms and signs.

In various embodiments, adjusting treatment comprises reducing the dose and/or frequency of treatment or ceasing administration of treatment, wherein each possibility represents a separate embodiment of the invention. Yet in other embodiments, adjusting treatment comprises replacing the treatment given to the subject with a milder treatment associated with fewer side effects. According to certain advantageous embodiments, treatment adjustment in subjects identified as having SLE resolution may include maintaining the subject without any SLE treatment while keeping said subject asymptomatic.

At present, the therapeutic paradigm in SLE involves a choice among multiple anti-inflammatory and immunosuppressive agents to reduce disease activity and limit acute and cumulative organ damage. SLE may be classified as mild (e.g., fever, arthritis, pleurisy, pericarditis, headache, rash) or severe (e.g., hemolytic anemia, thrombocytopenic purpura, massive pleural and pericardial involvement, significant renal damage, acute vasculitis of the extremities or GI tract, florid CNS involvement, diffuse alveolar hemorrhage). Mainstay treatments in SLE can include antimalarials, nonsteroidal anti-inflammatory drugs (NSAIDs), and low doses of corticosteroids for less severe disease. These modalities are helpful in treatment of mild symptoms such as arthralgias and musculoskeletal cutaneous manifestations. For more severe disease, corticosteroids and cytotoxic and immunosuppressive agents are used in patients with significant organ involvement and severe cutaneous manifestations. The antimalarial drug hydroxychloroquine, for example, is indicated for all patients with SLE regardless of disease severity because it decreases disease flares and decreases mortality.

NSAIDs inhibit the generation of prostaglandins by blocking cyclooxygenase enzymes, COX-1 and COX-2. Prostaglandins are mediators of inflammation and pain but also have important roles in maintenance of normal body functions including protection from stomach acid, maintenance of kidney blood flow, and contributing to platelet stickiness and vascular function. The major effect of these agents is to reduce acute inflammation thereby decreasing pain and improving function. All of these drugs also have mild to moderate analgesic properties independent of their anti-inflammatory effect. However these drugs alone do not change the course of the disease of rheumatoid arthritis or prevent joint destruction.

There are a large number of NSAIDs, and at full dosages all are potentially equally effective. Likewise, the toxicities of the currently available NSAIDs are similar. Many different NSAIDS are available, some over the counter including ibuprofen (Advil ®, Motrin®, Nuprin ®) and naproxen (Alleve®) and many others are available by prescription including meloxicam (Mobic®), etodolac (Lodine®), nabumetone (Relafen®), sulindac tolementin (Tolectin®), choline magnesium salicylate (Trilasate®), diclofenac (Cataflam®, Voltaren®, Arthrotec®), diflusinal (Dolobid®), indomethacin (Indocin®), ketoprofen (Orudis®, Oruvail®), meloxicam (Mobic®), oxaprozin (Daypro®), and piroxicam (Feldene®). Longer acting NSAIDs that allow daily or twice daily dosing may improve compliance. The NSAID class also includes drugs known as COX-2 inhibitors that are also effective in controlling inflammation, e.g. celecoxib, Celebrex®; etoricoxib, Arcoxia®; lumiracoxib, Prexige®. These drugs were designed to decrease the gastrointestinal risk of NSAIDS, but concerns of possible increases in cardiovascular risk with these agents has led to the withdrawal of two of these drugs from the market (rofecoxib, Vioxx®; valdecoxib, Bextra®).

NSAID doses for the treatment of SLE are known in the art. For example, ibuprofen may be used as needed or in doses up to 3000 mg a day, and naproxen is typically used as 500 mg twice a day.

Corticosteroids (such as prednisone; methylprednisolone, Medrol®) have both anti-inflammatory and immunoregulatory activity. They can be given orally, intravenously, intramuscularly or can be injected directly into the joint. In mild disease, prednisolone is given in doses starting at 0.1-0.3 mg/kg/day followed by a gradual tapering dose regimen according to clinical response. The dose rises to 0.4-0.6 mg/kg/day in moderate disease and as high as 0.7-1.5 mg/kg/day in very severe disease. At such high doses, pulse therapy with intravenous (IV) methylprednisolone (MP; 500-1000 mg on one to three occasions) is deemed by many physicians to be safer with fewer associated side effects. IV therapy is considered in patients that have not responded to oral therapy and/or have serious manifestations of SLE such as lupus nephritis, neuropsychiatric disease, severe refractory thrombocytopenia, hemolytic anemia, severe vasculitis and cardiopulmonary disease.

In patients with moderate-to-severe disease who require 10 mg prednisolone/day or more to manage their disease, other immunosuppressive agents are typically added to reduce the steroid requirements, reduce inflammation and ultimately organ damage.

For example the complication with the strongest evidence for efficacy is lupus nephritis. Methylprednisolone 1 g by slow (1-h) IV infusion on 3 successive days is often the initial treatment. Then, prednisone given in doses of 40 to 60 mg po once/day can be maintained, the dose may vary according to the manifestation of SLE. Cyclophosphamide or mycophenolate mofetil (especially in blacks) is usually also used for induction therapy. In severe renal involvement, cyclophosphamide is usually given in intermittent IV pulses instead of daily oral doses; e.g., about 500 mg to 1 g/m² IV (together with mesna and fluid loading to prevent drug-associated cystitis) monthly for 6 mo and then once q 3 mo for 18 mo (less frequently if there is renal or hematologic toxicity).

Cyclophosphamide (Cytoxan®) is a potent immunosuppressive agent, acting as an alkylating agent, which causes cell death at any stage of the cell cycle. It also depletes both B and T cells, hence reducing the production of pathogenic auto-antibodies. It may be given orally or intravenously.

Azathioprine (Imuran®) is an immunosuppressive agent commonly used for the induction of remission and as a steroid-sparing agent in mild-to-moderate disease. It works by affecting cell-mediated and humoral immune responses via the inhibition of lymphocyte proliferation, reduction in antibody production and suppression of natural killer cell activity. In severe disease, it is used as maintenance therapy and data from lupus nephritis trials show significant improvement in disease activity following induction therapy with cyclophosphamide or mycophenolate mofetil. It may be associated with gastrointestinal side effects, such as nausea, vomiting and diarrhea, occasionally severe enough to lead to drug withdrawal. Mild transaminitis may occur and reversible bone marrow suppression is not uncommon.

Antimalarial drugs have been used in rheumatology for the treatment of SLE for many years. These include e.g. chloroquine, mepacrine (quinacrine/atabrine) and hydroxychloroquine. Chloroquine sulphate and phosphate are associated with the greatest risk of ocular toxicity and are now rarely prescribed. Mepacrine may be useful for lupus-induced skin rashes but it has little effect on other manifestations. Exemplary Hydroxychloroquine dose is 5 mg/kg po of body weight once/day. Alternatives include e.g. chloroquine 250 mg po once/day and quinacrine 50 to 100 mg po once/day.

Other biological agents and immunomodulators which may be used include for example monoclonal antibodies targeting several surface molecules on B cells, to reduce the formation of auto-antibodies. Such exemplary drugs include rituximab (anti-CD20), ocrelizumab (humanized anti-CD20), belimumab (anti-BAFF/BLyS), atacicept (anti-BLys/APRIL) and epratuzumab (humanized anti-CD22). In addition, other key cell-surface markers have been developed to interfere with costimulatory molecules such as cytotoxic T lymphocyte antigen 4 (abatacept). In addition, Leflunomide (original brand name Arava) is an immunosuppressive pyrimidine synthesis inhibitor that works by inhibiting dihydroorotate dehydrogenase. Leflunomide is an immunomodulatory drug that inhibits the reproduction of rapidly dividing cells, especially lymphocytes. In addition, TNF-α inhibitors, e.g. Etanercept or Infliximab, may be used in some cases.

According to various embodiments, treatment adjustment may be performed for the following exemplary treatments: nonsteroidal anti-inflammatory drugs (NSAIDs), corticosteroids, immunosuppressants, hydroxychloroquine, cyclophosphamide, immunomodulators, and TNF-α inhibitors. In other embodiments, said treatment is selected from the group consisting of: NSAIDs, corticosteroids, myfortic, Methotrexate, Imuran, Abatacept, Hizentra, Gammagard, Octagam, Privigen, Arava, Plaquenil, Cyclophosphamide, Benlysta, Rituximab and Orenica. Each possibility represents a separate embodiment of the invention.

In a particular embodiment, said treatment comprises corticosteroid treatment. In another particular embodiment, said treatment adjustment comprises discontinuing administration of corticosteroids. In another particular embodiment, said treatment adjustment comprises reducing the dose and/or frequency of administration of corticosteroids (e.g. from 0.7-1.5 mg/kg/day to 0.4-0.6 mg/kg/day or from 0.4-0.6 mg/kg/day to 0.1-0.3 mg/kg/day).

The following examples are presented in order to more fully illustrate some embodiments of the invention. They should, in no way be construed, however, as limiting the scope of the invention.

### EXAMPLES

### Materials and Methods

The antigens used in the experiments are listed in Table 1 below:

TABLE 1
Antigens

| | SEQ | | |
|---|---|---|---|
| | ID | Full name/ | Manufacture |
| Antigen | NO: | amino acid or oligonucleotide sequence | (catalog no.) |
| DNAse I | 1 | Deoxyribonuclease I | AKRON |
| | | MRGMKLLGALLALAALLQGAVSLKIAAFNIQTFGETKMSNAT | biotech |
| | | LVSYIVQILSRYDIALVQEVRDSHLTAVGKLLDNLNQDAPDT | (AK3778) |
| | | YHYVVSEPLGRNSYKERYLFVYRPDQVSAVDSYYYDDGCEPC | |
| | | GNDTFNREPAIVRFFSRFTEVREFAIVPLHAAPGDAVAEIDA | |
| | | LYDVYLDVQEKWGLEDVMLMGDFNAGCSYVRPSQWSSIRLWT | |
| | | SPTFQWLIPDSADTTATPTHCAYDRIVVAGMLLRGAVVPDSA | |
| | | LPFNFQAAYGLSDQLAQAISDHYPVEVMLK | |
| ssDNA | | single stranded DNA (calf) | Sigma |
| | | | (D8899) |
| Histone | | Histone from calf thymus | Sigma |
| IIIS | | (Type III-S) | (H5505) |
| Collagen | 2 | Type III collagen | AKRON |
| III | | MMSFVQKGSWLLLALLHPTIILAQQEAVEGGCSHLGQSYADR | biotech |
| | | DVWKPEPCQICVCDSGSVLCDDIICDDQELDCPNPEIPFGEC | (AK9914) |
| | | CAVCPQPPTAPTRPPNGQGPQGPKGDPGPPGIPGRNGDPGIP | |
| | | GQPGSPGSPGPPGICESCPTGPQNYSPQYDSYDVKSGVAVGG | |
| | | LAGYPGPAGPPGPPGPPGTSGHPGSPGSPGYQGPPGEPGQAG | |
| | | PSGPPGPPGAIGPSGPAGKDGESGRPGRPGERGLPGPPGIKG | |
| | | PAGIPGFPGMKGHRGFDGRNGEKGETGAPGLKGENGLPGENG | |
| | | APGPMGPRGAPGERGRPGLPGAAGARGNDGARGSDGQPGPPG | |
| | | PPGTAGFPGSPGAKGEVGPAGSPGSNGAPGQRGEPGPQGHAG | |
| | | AQGPPGPPGINGSPGGKGEMGPAGIPGAPGLMGARGPPGPAG | |
| | | ANGAPGLRGGAGEPGKNGAKGEPGPRGERGEAGIPGVPGAKG | |
| | | EDGKDGSPGEPGANGLPGAAGERGAPGFRGPAGPNGIPGEKG | |
| | | PAGERGAPGPAGPRGAAGEPGRDGVPGGPGMRGMPGSPGGPG | |
| | | SDGKPGPPGSQGESGRPGPPGPSGPRGQPGVMGFPGPKGNDG | |
| | | APGKNGERGGPGGPGPQGPPGKNGETGPQGPPGPTGPGGDKG | |
| | | DTGPPGPQGLQGLPGTGGPPGENGKPGEPGPKGDAGAPGAPG | |
| | | GKGDAGAPGERGPPGLAGAPGLRGGAGPPGPEGGKGAAGPPG | |
| | | PPGAAGTPGLQGMPGERGGLGSPGPKGDKGEPGGPGADGVPG | |
| | | KDGPRGPTGPIGPPGPAGQPGDKGEGGAPGLPGIAGPRGSPG | |
| | | ERGETGPPGPAGFPGAPGQNGEPGGKGERGAPGEKGEGGPPG | |
| | | VAGPPGGSGPAGPPGPQGVKGERGSPGGPGAAGFPGARGLPG | |
| | | PPGSNGNPGPPGPSGSPGKDGPPGPAGNTGAPGSPGVSGPKG | |
| | | DAGQPGEKGSPGAQGPPGAPGPLGIAGITGARGLAGPPGMPG | |
| | | PRGSPGPQGVKGESGKPGANGLSGERGPPGPQGLPGLAGTAG | |
| | | EPGRDGNPGSDGLPGRDGSPGGKGDRGENGSPGAPGAPGHPG | |
| | | PPGPVGPAGKSGDRGESGPAGPAGAPGPAGSRGAPGPQGPRG | |
| | | DKGETGERGAAGIKGHRGFPGNPGAPGSPGPAGQQGAIGSPG | |
| | | PAGPRGPVGPSGPPGKDGTSGHPGPIGPPGPRGNRGERGSEG | |
| | | SPGHPGQPGPPGPPGAPGPCCGGVGAAAIAGIGGEKAGGFAP | |
| | | YYGDEPMDFKINTDEIMTSLKSVNGQIESLISPDGSRKNPAR | |
| | | NCRDLKFCHPELKSGEYWVDPNQGCKLDAIKVFCNMETGETC | |
| | | ISANPLNVPRKHWWIDSSAEKKHVWFGESMDGGFQFSYGNPE | |
| | | LPEDVLDVHLAFLRLLSSRASQNITYHCKNSIAYMDQASGNV | |
| | | KKALKLMGSNEGEFKAEGNSKFTYTVLEDGCTKHTGEWSKTV | |
| | | FEYRTRKAVRLPIVDIAPYDIGGPDQEFGVDVGPVCFL | |
| Ul | | Small Nuclear Ribonucleoprotein 70 kDa Human | Prospec (pro- |
| snRNP | | Recombinant | 445) |
| Ro52 | 3 | 52 kDa Ro protein | Prospec (PRO- |
| | | MASAARLTMMWEEVTCPICLDPFVEPVSIECGHSFCQECISQ | 328) |
| | | VGKGGGSVCPVCRQRFLLKNLRPNRQLANMVNNLKEISQEAR | |
| | | EGTQGERCAVHGERLHLFCEKDGKALCWVCAQSRKHRDHAMV | |
| | | PLEEAAQEYQEKLQVALGELRRKQELAEKLEVEIAIKRADWK | |
| | | KTVETQKSRIHAEFVQQKNFLVEEEQRQLQELEKDEREQLRI | |
| | | LGEKEAKLAQQSQALQELISELDRRCHSSALELLQEVIIVLE | |
| | | RSESWNLKDLDITSPELRSVCHVPGLKKMLRICAVHITLDPD | |
| | | TANPWLILSEDRRQVRLGDTQQSIPGNEERFDSYPMVLGAQH | |
| | | FHSGKHYWEVDVTGKEAWDLGVCRDSVRRKGHFLLSSKSGFW | |
| | | TIWLWNKQKYEAGTYPQTPLHLQVPPCQVGIFLDYEAGMVSF | |
| | | YNITDHGSLIYSFSECAFTGPLRPFFSPGFNDGGKNTAPLTL | |
| | | CPLNIGSQGSTDY | |
| Sm | | Smith antigen | US Biological |
| | | | (s1014-29F) |
| Apo- | 4 | Recombinant Human Apo-SAA | Peprotec (300- |
| SAA | | MRSFFSFLGE AFDGARDMWR AYSDMREANY | 13) |
| | | IGSDKYFHAR GNYDAAKRGP GGVWAAEAIS | |
| | | NARENIQRFF GRGAEDSLAD QAANEWGRSG | |
| | | KDPNHFRPAG LPEKY | |
| H2a | 5 | Histone H2A Human | Sigma |
| | | MSGRGKQGGKARAKAKTRSSRAGLQFPVGRVHRLLRKGNYAE | (H9250) |
| | | RVGAGAPVYLAAVLEYLTAEILELAGNAARDNKKTRIIPRHL | |
| | | QLAIRNDEELNKLLGKVTIAQGGVLPNIQAVLLPKKTESHHK | |
| | | AKGK | |
| Oligo21 | 6 | TTA GGG TTA GGG TTA GGG TTA GGG | |

The clinical and demographic data of the subjects in this study are tabulated in Table 2 below:

TABLE 2
Clinical and demographic data

| | | | SLE Patient | SLE Patient |
|---|---|---|---|---|
| | Healthy | SLE | Pairs Time Post | Pairs Time Post |
| | Controls | Patients | Diagnosis < 10 years | Diagnosis > 10 years |
| Sample Demographics | N = 51 | N = 50 | (N = 84) | (N = 97) |
| Age in years Mean (±SD) | 37.8 (11.2) | 36.6 (11.8) | 37.2 (13.7) | 47.8 (12.3) |
| Mean time post diagnosis | — | 1 (0.96) | 3.92 (2.86) | 18.5 (8.3) |
| Gender | | | | |
| Female | 100% | 100% | 93% | 97.90% |
| Male | | | 7% | 2.10% |
| Ethnic category N (%) | | | | |
| Afro-American | 21 (41.2) | 23 (46.0) | 28 (50%) | 30 (52.6%) |
| White non-Hispanic | 15 (29.4) | 15 (30.0) | 3 (5.4%) | 15 (26.3%) |
| Indian/Asian/Middle Eastern | 6 (11.8) | 1 (2) | 2 (3.6%) | 2 (3.5%) |
| White Hispanic | 8 (15.7) | 9 (18.0) | 23 (41.1%) | 10 (17.5%) |
| Other | 1 (2.0) | 2 (4.0) | 28 unknown | 40 unknown |

Autoantibody patterns in two serum samples from SLE patients were analyzed at various times after diagnosis (n=181 patients, 362 samples). SLE serum samples and clinical information were obtained from the repositories of four independent, major lupus centers, and were approved by each respective IRB. Samples were collected from females not known to be pregnant (96%) and males (4%), between 17-84 years of age at the time of first sample collection. For 84 patients, both samples were obtained at less than 10 years after diagnosis (mean time post diagnosis for T1 sample was 3.92±2.86 years). In 97 cases, at least one sample was obtained at 10 years or more after diagnosis (mean time post diagnosis for T1 sample was 18.52±8.34 years). All the patients fulfilled four or more criteria established by the American College of Rheumatology (ACR). SLEDAI scores at the time of blood draw ranged from 0 to 22; differences between the SLEDAI scores in individual patients ranged from 2 to 20 points. 65.2% of pairs (n=118) manifested a decrease in SLEDAI score at time point 2 (T2) relative to time point 1 (T1), and 34.3% (n=62) showed an increase; SLEDAI data was not available for one patient. The T1-T2 time interval ranged from several weeks to 12 years (mean 1.54±2.31 years).

Microarray preparation: Glass slides were coated with an epoxy silane organic layer, using a YES1224p oven (Yield Engineering Systems, CA, USA). After coating, the slides were packed into slide boxes and vacuum-sealed until printing. Antigens were printed on the coated slides using a Scienion S11 non-contact arrayer (Scienion AG, Germany). The 8 frames slides were blocked with 250 uL of 1% casein (Sigma) and incubated on a rocker (1 h, room temperature, 17 rpm), blocking solution was removed and the diluted serum was added subsequently as described below.

Antigen Array: Each slide includes 8 identical wells. Testing was performed using 8 well-frame.

Serum testing: The slides were allowed to reach room temperature and serum specimens were fully thawed before testing. Serum samples were diluted 1:75 in freshly filtered 1% casein. Diluted serum (150 μl) was dispensed in each well and then incubated (1 h, 37° C.). Each well was then washed with 250 uL PBS, PBS-T, and PBS (5 minutes, room temperature, 17 rpm).

Secondary antibody application: Slides were incubated with goat anti-human IgG-Cy3 (Jackson ImmunoResearch Laboratory) and goat anti-human IgM-Dy5 (Jackson ImmunoResearch Laboratory) diluted 1:2300 in 1% casein in PBS. 150 μl of mixed secondary antibody was dispensed in each well and then incubated (1 hour, 37° C., 8 rpm). Each well was then washed with 250 uL PBS, PBS-T, PBS (5 minutes, room temperature, 17 rpm) and ROW (1 minute, room temperature, 17 rpm). Slides are then centrifuged for 10 min at 2500 rpm at room temperature (23° C.).

Slide scanning and data processing: The slides were scanned at two wavelengths (532 nm and 633 nm) using an Agilent scanner (Agilent Technologies, Santa Clara, Calif.) and images were extracted using the GenePix 7.0 feature extraction (FE) software (Molecular Devices, Sunnyvale, Calif.), using default settings. All scanned images were preprocessed.

Data preprocessing involved these major steps: subtraction of background, data transformation, removal of outliers, combination of replicates, adjustment of overall intensity per slide and correction of print lot effect. Specifically, the pre-processing procedure included the following steps:

Determining representation of each spot through subtraction of background intensity from the foreground intensity; imputing negative values with low intensity spots, as negative spots have no meaning in the context of the platform; performing log transformation on each spot and combining spot replicates per array, wherein outliers spots were removed based on Grubb's test; adjusting the mean per array per channel, wherein the mean per array was calculated based on a set of antigens per channel, and the mean may then be adjusted against a constant value and the correction was applied to all antigens; and correcting for print lot and test session effect using reference sera, wherein the correction was performed per test session per print lot, by subtraction of reference serum intensities from corresponding antigens in order to express all intensities as relative to the reference serum.

### Example 1

The SLE test retains sensitivity up to 10 years from diagnosis of SLE.

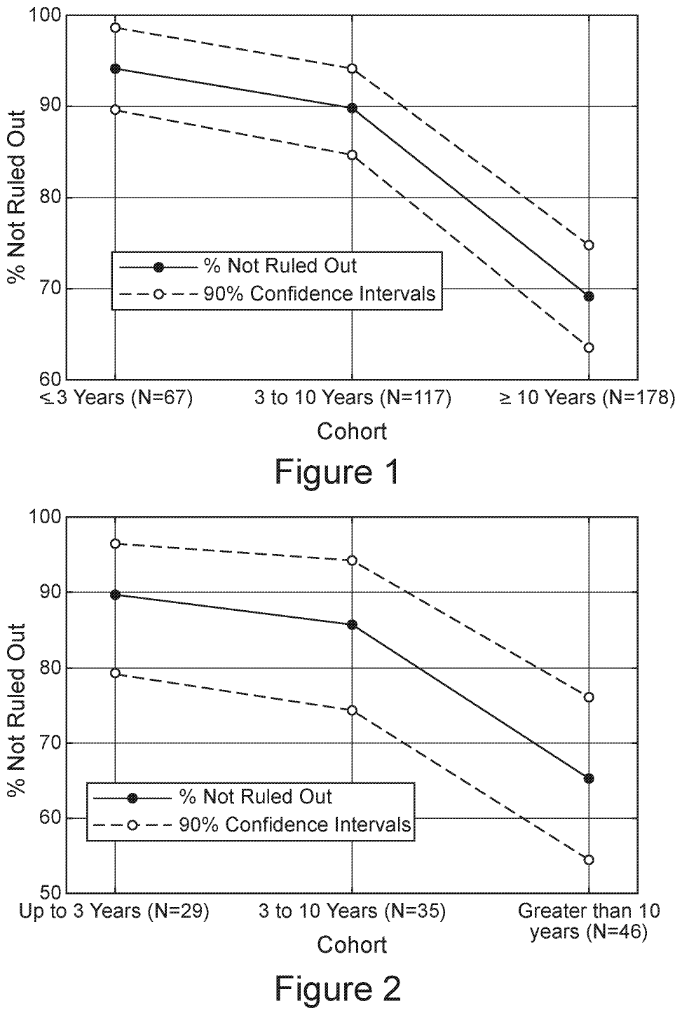

To learn if the SLE test results are affected by the time elapsed since diagnosis, the percent of subjects positively identified by the SLE test (hereinafter designated “Not Ruled-Out”) were examined in three groups of SLE patient samples: those samples tested within 3 years of diagnosis (n=116); those tested between 3 and 10 years of diagnosis (n=117); and those tested at 10 years or thereafter (n=178) after diagnosis. FIG. 1 shows that at or above 3 years post diagnosis, 90% of the SLE patients were designated as Not Ruled-Out; this fraction of patients Not Ruled-Out is similar to what was observed with the original SLE test validation cohort. The percent of patients Not Ruled Out drops slightly from 3 to 10 years after diagnosis. Surprisingly, at 10 or more years after diagnosis, the fraction of those Not Ruled-Out decreased to about 69% (FIG. 1), a shift in the autoimmune signature suggesting that these patients more closely resembled the healthy controls who were Ruled out in the original validation cohort (specificity: 75%).

The change from Not Ruled-Out to Ruled-Out could not be attributed to serum storage time—the serum storage times of each of the three groups were not significantly different (4.04±3.46, 5.61±4.05 and 6.07±3.44 years respectively). The ages at diagnosis were also similar (35±14, 32±14, and 29±12 years respectively) indicating that we were not looking at patients with late-onset SLE.

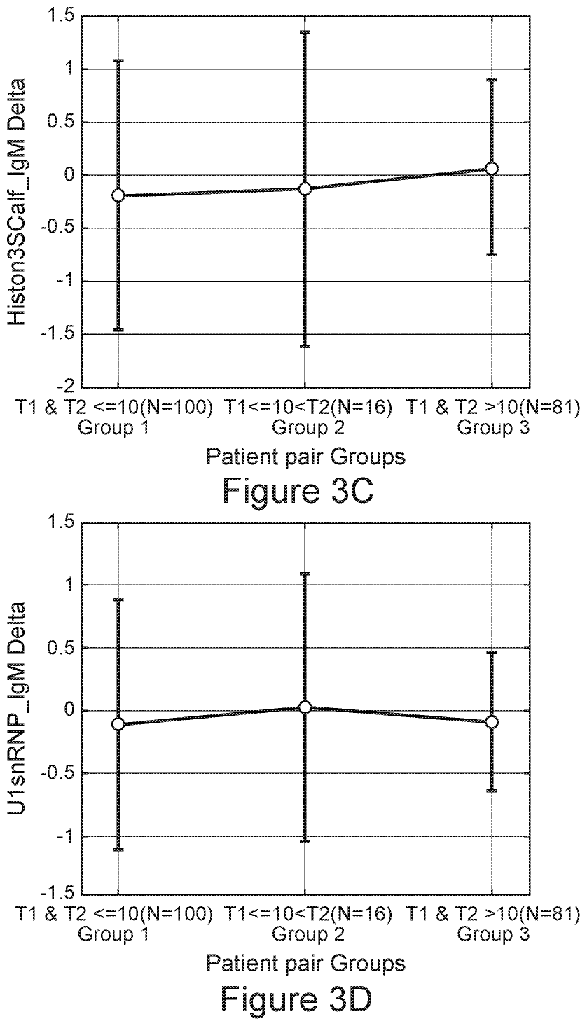

As can be seen in FIG. 3, the change was not detectable as a change in an individual marker of the markers that constitute the SLE test classifier algorithm, highlighting the importance of the integrated autoimmune signature in the determination of the test results.

Thus, while the serological profile of SLE patients is stable for the first approximately 10 years after diagnosis, a change in the patient autoimmune profile may occur in later years, leading to a designation of Ruled-Out in greater than 30% of SLE patient samples.

### Example 2

The SLE signature is independent of disease activity as expressed by SLEDAI

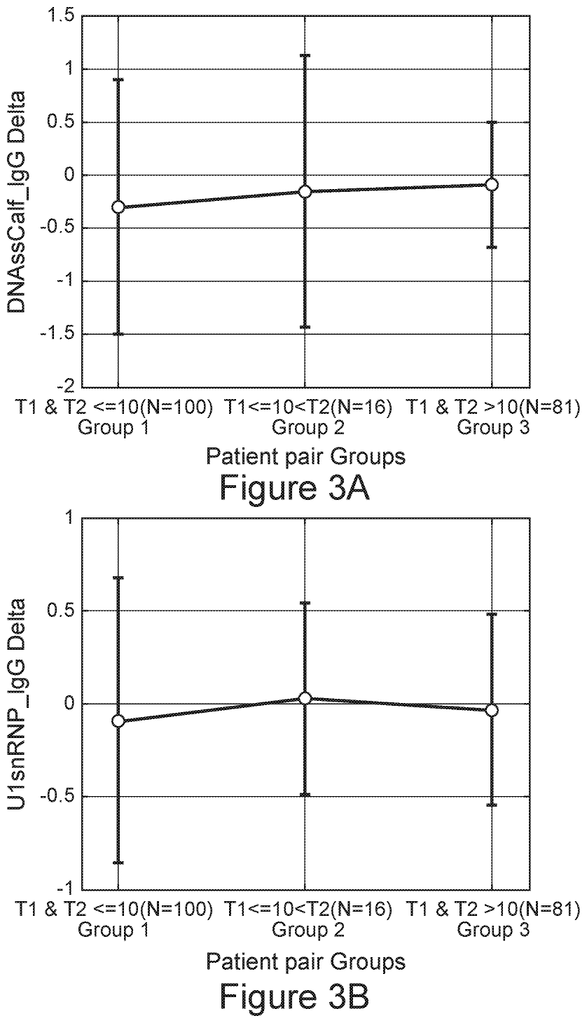

SLEDAI scores were available for both the original validation cohort and the pairs cohort described above. In the case of asymptomatic (SLEDAI=0) patients, the sensitivity of the SLE test classifier as a function of time post disease diagnosis parallels the data from the full cohort. The three groups, shown in FIG. 2, contained somewhat fewer subjects (29, 35 and 46 each), but the results were essentially similar. Despite SLEDAI scores of 0, about 90% of the patients manifested a test designation of Not Ruled-Out at 3 years or between 3 and 10 years since diagnosis. Similar to the full cohort, the percent of subjects with designations of SLE Not Ruled-Out fell to about 65% after 10 or more years in the SLEDAI=0 subset of patients.

The results of the SLE test were not influenced by the SLEDAI score. During the first 10 years following diagnosis (see FIG. 1), when the SLE test successfully identifies 90% of SLE patients as Not Ruled-Out, the patients exhibited a wide range of SLEDAI scores—between 0 and 19. Likewise, the range of SLEDAI scores in patients who remained Not Ruled-Out 10 or more years after diagnosis was between 1 and 18.

The lack of correlation between SLE test status and SLEDAI scores suggests that the six autoantibody reactivities included in the SLE test are not likely to be directly involved in SLE clinical pathology. The SLE test signature may rather reflect an underlying autoantibody signature that distinguishes SLE from health.

### Example 3

Reduction in the frequency of the Lupus Signature after 10 Years

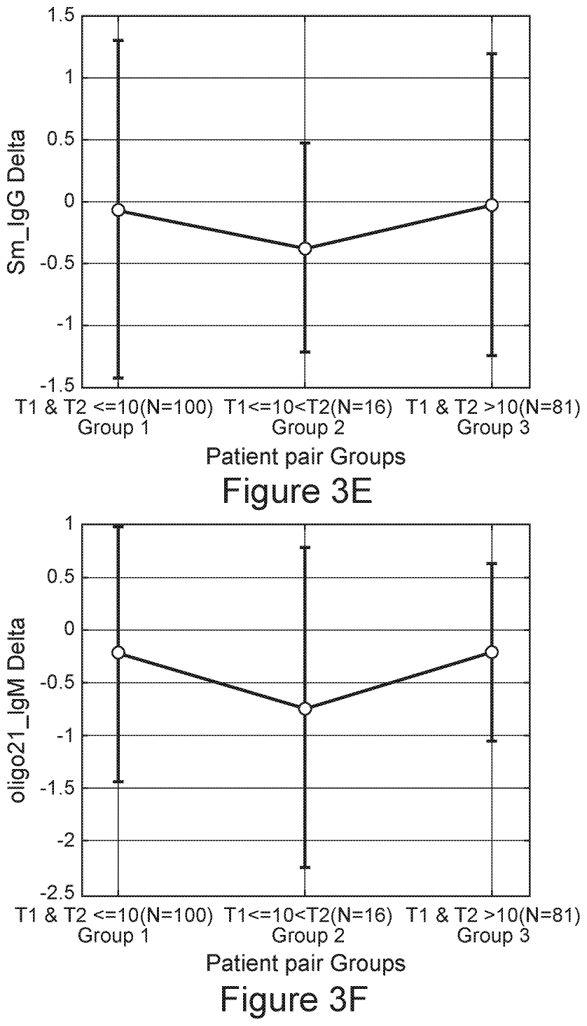

The SLE signature was developed to distinguish between SLE patients and healthy individuals. Using the threshold established during the validation of the test, an increase in the frequency of SLE patients who are Ruled Out with the SLE test was surprisingly identified when more than 10 years have elapsed since diagnosis. Looking at the SLE score itself, a downward trend in the SLE score can be seen at greater times post diagnosis (FIG. 4). The mean numerical scores of 0.9 to 0.8 at 3 years and at 3-10 years correspondingly fell to a mean of less than 0.5 at 10 or more years (p=4.1E⁻¹⁰). Thus the increase in subjects developing a Ruled-Out designation was accompanied by a significant fall in mean numerical score. FIG. 4 shows the shift in numerical SLE signature scores in the patient subsets categorized according to the time since SLE diagnosis. The median numerical scores of 0.89 (IQR 0.51) and 0.83 (IQR 0.5) in disease less than 3 years, and 3 to 10 years respectively, fell to a median of less than 0.44 (IQR 0.78) at 10 or more years after diagnosis (p=1.3E⁻⁰⁹). Thus, there is both an increase in the number of subjects developing an SLE Ruled-Out designation and a general decrease in the mean SLE test scores after 10 or more years.

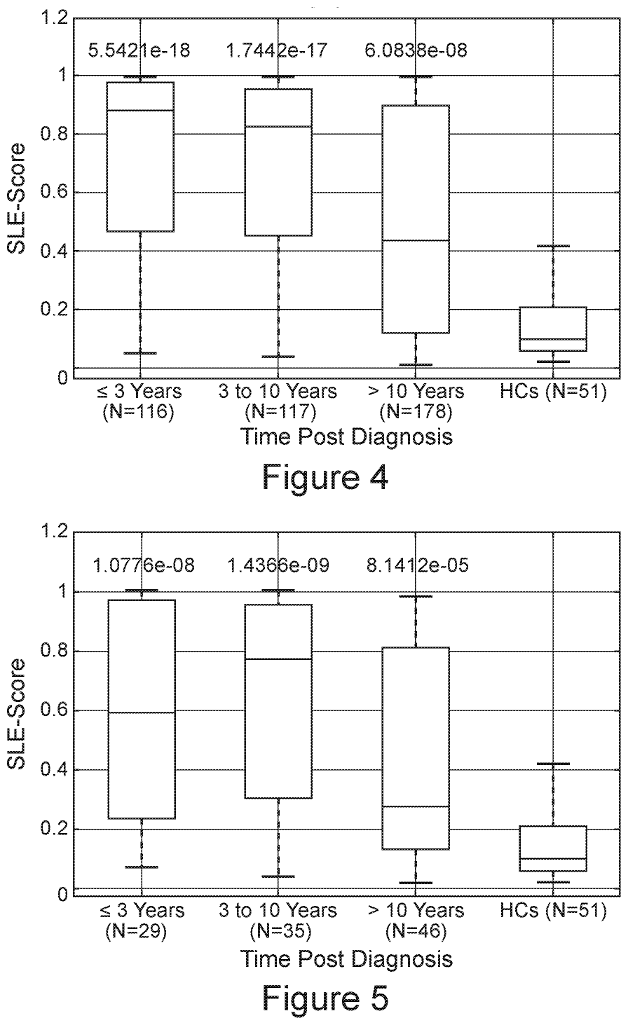

A similar and possibly more pronounced trend may be seen in the subset of SLEDAI=0 patients (FIG. 5). To dissociate the change in immune profile from potential variations in disease activity, patients with low disease activity were separately examined. FIG. 5 shows a waning of the SLE-key Rule-Out test scores in asymptomatic subjects manifesting SLEDAI scores of 0; after 10 years the mean numerical score of asymptomatic SLE patients approached that of healthy individuals.

These results suggest that in a subset of SLE patients, the autoimmune signature characteristic of SLE may be evolving over time back to a signature more typically observed in healthy individuals.

### Example 4

The SLE signature is replaced by the healthy signature over time in a subset of patients

In the larger population, using a cutoff of 10 years, significant changes in the autoimmune profile of the diagnosed SLE patients were surprisingly observed. Next, individual patients in the subset of paired patient samples drawn at different times post diagnosis were examined, to identify patients demonstrating a significant decrease in SLE score from T1 to T2. 31/181 patients (17%) met these criteria, wherein the time post diagnosis for the T2 sample in these patients ranged between 0 and 46 years (mean time post diagnosis: 12.6 years). Among these 31 patients, 16 were asymptomatic (SLEDAI=0) at T2. For these patients, the time post diagnosis at T2 ranged between 2.6 and 46 years with a mean time post diagnosis of 17.9 years. This contrasted with the group of patients who—while demonstrating a downward trend in their SLE score, they still had SLEDAI scores >0. In these patients, time post diagnosis for T2 ranged between 0 and 25 years with a mean of 7 years elapsed since diagnosis.

### Example 5

SLE serological manifestations and medication use in the different patient populations

SLE serological manifestations and medication use were further recorded, and compared between patients grouped according to the time since diagnosis. Table 3 below shows the prevalence of high anti-double stranded (ds) DNA antibodies, low serum C3, and low serum C4 in the different groups, all defined as values outside the normal range in each institution at the time of the blood draw, respectively. Information regarding usage of immunosuppressant medications, corticosteroids, and anti-malarial drugs is provided in Table 4 below.

As can be determined from Table 3, patients in Group 1 (where both samples in the longitudinal study were obtained within 10 years of diagnosis) manifested a significantly higher prevalence of abnormal anti-dsDNA antibodies and serum C4 complement levels. In addition, when analyzing a possible relationship between medication use and the SLE score, no increased incidence of a positive Rule-Out score (an excluded lupus diagnosis) was observed with higher usage of immunosuppression or immunomodulation. Rather, the opposite was surprisingly observed—in patient pairs in which at least one of the samples was obtained more than 10 years after the diagnosis (Groups 2 and 3), the prevalence of corticosteroid use was significantly decreased (Table 4); these patients apparently could be managed with less corticosteroids.

Thus, an attenuation of the autoimmune process over time was surprisingly identified in a selected patient population, which is also reflected in the increased incidence of a positive SLE Rule-Out test as demonstrated herein. These changes were not reflected in the SLEDAI score, possibly because serological changes often precede changes in clinical manifestations in lupus and/or as the SLEDAI score may lack sufficient sensitivity.

TABLE 3
Serological results

| | | High | Low | Low |
|---|---|---|---|---|
| | | anti-dsDNA | C3 | C4 |
| Group 1: | N | 47 | 74 | 73 |
| T1 & T2 ≤ 10 | Positive | 40% | 22% | 32% |
| years | Negative | 45% | 57% | 44% |
| Group 2: | N | 12 | 16 | 16 |
| T1 < 10; | Positive | 8%* | 13% | 19% |
| T2 > 10 | Negative | 75% | 69% | 63% |
| Group 3: | N | 38 | 66 | 66 |
| T1 & T2 > 10 | Positive | 16%* | 12% | 15%** |
| years | Negative | 68% | 74% | 76% |
| Group 2 + 3 | N | 50 | 82 | 82 |
| (combined) | Positive | 14%** | 12% | 16%** |
| | Negative | 70% | 73% | 73% |

N = number of pairs with data available at both time points
Positive = patients who were positive at both time points
Negative = patients who were negative at both time points
All comparisons are versus Group 1 using Fisher's exact test; significant comparisons are bolded.
*p < 0.05;
**p < 0.01

TABLE 4
Medication use

| | | |
|---|---|---|
| Group 1: | N | 82 |
| T1 & T2 ≤ 10 years | Immunosuppressants | 26 (32%) |
| | Corticosteroids | 52 (63%) |
| | Anti-malarials | 21 (26%) |
| Group 2: | N | 16 |
| T1 < 10; T2 > 10 | Immunosuppressants | 4 (25%) |
| | Corticosteroids | 4 (25%)** |
| | Anti-malarials | 1 (6%) |
| Group 3: | N | 77 |
| T1 & T2 > 10 years | Immunosuppressants | 27 (35%) |
| | Corticosteroids | 31 (40%)** |
| | Anti-malarials | 24 (31%) |
| Group 2 + 3 | N | 93 |
| (combined) | Immunosuppressants | 31 (33%) |
| | Corticosteroids | 35 (38%)*** |
| | Anti-malarials | 25 (27%) |

N = total number of pairs in the group
Values are for patient pairs receiving medication at T1 & T2
Immunosuppressants: Cyclophosphamide, azathioprine, cyclosporine, tacrolimus, methotrexate, rituximab
Corticosteroids: Prednisone or methylprednisolone
Anti-malarials: Hydroxychloroquine or quinacrine
All comparisons are versus Group 1 using Fisher's exact test; significant comparisons are bolded.
**p < 0.01;
***p < 0.001

In summary, the results observed both in the SLE patient population and in individual patients over time suggests that some patients may resolve their autoimmune disease status and corresponding test signature, reverting to a signature consistent with that of a healthy individual. Tracking changes in SLE scores along with disease activity can help identify this patient population, who may be amenable for adjustment or termination of their SLE treatment regimen.

The improvement in serological activity and decrease in corticosteroid use during the longitudinal follow-up, correlated with the reduction in SLE signature, support the conclusion that these findings reflect attenuation of the autoimmune disease process over time in a selected, newly identified patient population, which is also reflected in the increased incidence of a positive SLE Rule-Out test. These finding further demonstrate the applicability of SLE treatment adjustment to the new patient population, identified according to the methods disclosed herein.

The foregoing description of specific embodiments and examples constructed according to the principles of the invention, will so fully reveal the general nature of the inventive concepts that others can, by applying current knowledge, readily modify and/or adapt for various applications such specific embodiments without undue experimentation and without departing from the generic concepts, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the principles of the invention.

1-28. (canceled)

29. A method for adjusting treatment in a subject having been diagnosed as having systemic lupus erythematosus (SLE) at least three years earlier, the method comprising the steps of:
(i) providing a first sample obtained from the subject at a first time point and a second sample obtained from the same subject at a second, subsequent time point;
(ii) exposing antibodies in each of the two samples to at least four antigens selected from the group consisting of: ssDNA, Sm, DNAse I, Histone III-S, Ro52, U1 snRNP, Collagen III, Apo-SAA, H2a, and Oligo21 to detect the respective reactivity patterns of the two samples to the at least four antigens;
(iii) calculating scores based on the reactivity patterns of the two samples by a supervised classification algorithm, wherein the lower the score the greater is the probability that the subject is not afflicted with SLE;
(iv) comparing the scores obtained for the two samples; and
(v) determining that the subject is amenable for treatment adjustment if there is a significant reduction of the score obtained for the second sample compared to the score obtained for the first sample.

30. The method of claim 29, wherein the treatment adjustment comprises reducing the dose and/or frequency of the treatment or ceasing administration of the treatment to the subject.

31. The method of claim 29, further comprising adjusting treatment in the subject determined to be amenable for treatment adjustment.

32. The method of claim 29, wherein the first time point precedes the second time point by at least ten years and/or wherein the subject has been diagnosed as having SLE at least ten years earlier.

33. The method of claim 29, wherein the subject is asymptomatic at the second time point.

34. The method of claim 29, wherein the supervised classification algorithm is selected from the group consisting of support vector machines (SVMs), logistic regression (LR), quadratic discriminant analysis (QDA), and linear discriminant analysis (LDA), and wherein the reactivity of antibodies comprises IgG reactivities, IgM reactivities, or a combination thereof.

35. The method of claim 29, wherein the reactivity pattern comprises reactivities of IgG antibodies to ssDNA, Sm, DNAse I, Ro52, and U1 snRNP, and reactivities of IgM antibodies to Histone III-S, and wherein the supervised classification algorithm is SVMs; or wherein the reactivity pattern comprises reactivities of IgG antibodies to ssDNA, U1 snRNP, Ro52, Collagen III, and Apo-SAA, and reactivities of IgM antibodies to Histone III-S, and wherein the supervised classification algorithm is LR; or
wherein the reactivity pattern comprises reactivities of IgG antibodies to ssDNA, U1 snRNP, Sm, Apo-SAA, and Ro52, and reactivities of IgM antibodies to H2a, and wherein the supervised classification algorithm is QDA; or
wherein the reactivity pattern comprises reactivities of IgG antibodies to ssDNA, U1 snRNP, and Sm, and reactivities of IgM antibodies to Histone III-S, U1 snRNP and Oligo21, and wherein the supervised classification algorithm is LDA.

36. The method of claim 29, wherein the sample is selected from the group consisting of a serum sample, a plasma sample, and a blood sample, and wherein the antigens are used in the form of an antigen probe set, an antigen array, or an antigen chip.

37. The method of claim 29, wherein the treatment is selected from the group consisting of: nonsteroidal anti-inflammatory drugs (NSAIDs), corticosteroids, immunosuppressants, hydroxychloroquine, cyclophosphamide, immunomodulators, and TNF-α inhibitors.

38. The method of claim 37, wherein the treatment is selected from the group consisting of: NSAIDs, corticosteroids, myfortic, Methotrexate, Imuran, Abatacept, Hizentra, Gammagard, Octagam, Privigen, Arava, Plaquenil, Cyclophosphamide, Benlysta, Rituximab, and Orenica.

39. The method of claim 29, wherein the scores are calculated in the range of 0 to 1 in which the lower the score the greater is the probability that the subject is not afflicted with SLE, and the significant reduction of the score obtained for the second sample compared to the score obtained for the first sample is of at least 0.1.

40. The method of claim 29, comprising the steps of:
(i) providing a first sample obtained from the subject at a first time point and a second sample obtained from the same subject at a second, subsequent time point, wherein the first time point precedes the second time point by at least ten years;
(ii) exposing antibodies in each of the two samples to a plurality of antigens selected from the group consisting of: ssDNA, Sm, DNAse I, Histone III-S, Ro52, U1 snRNP, Collagen III, Apo-SAA, H2a, and Oligo21 to detect the respective reactivity patterns of the two samples to the plurality of antigens, and calculating scores based on the reactivity patterns of the two samples, in which the lower the score the greater is the probability that the subject is not afflicted with SLE, using a supervised classification algorithm, wherein:
a. the reactivity pattern comprises reactivities of IgG antibodies to ssDNA, U1 snRNP, Ro52, Collagen III, and Apo-SAA, and reactivities of IgM antibodies to Histone III-S, and the supervised classification algorithm is logistic regression (LR); or
b. the reactivity pattern comprises reactivities of IgG antibodies to ssDNA, U1 snRNP, Sm, Apo-SAA, and Ro52, and reactivities of IgM antibodies to H2a, and the supervised classification algorithm is quadratic discriminant analysis (QDA); or
c. the reactivity pattern comprises reactivities of IgG antibodies to ssDNA, U1 snRNP, and Sm, and reactivities of IgM antibodies to Histone III-S, U1 snRNP, and Oligo21, and the supervised classification algorithm is linear discriminant analysis (LDA); or
d. the reactivity pattern comprises reactivities of IgG antibodies to ssDNA, Sm, DNAse I, Ro52, and U1 snRNP, and reactivities of IgM antibodies to Histone III-S, and the supervised classification algorithm is support vector machines (SVMs);
(iii) comparing the scores obtained for the two samples; and
(iv) determining that the subject is amenable for treatment adjustment if there is a reduction of at least 0.1 in the score obtained for the second sample compared to the score obtained for the first sample.

41. The method of claim 29, comprising the steps of:
(i) providing a first sample obtained from the subject at a first time point and a second sample obtained from the same subject at a second, subsequent time point, wherein the first time point precedes the second time point by at least ten years;
(ii) exposing antibodies in each of the two samples to a plurality of antigens selected from the group consisting of: ssDNA, Sm, DNAse I, Histone III-S, Ro52, U1 snRNP, Collagen III, Apo-SAA, H2a, and Oligo21 to detect the respective reactivity patterns of the two samples to the plurality of antigens, and calculating scores based on the reactivity patterns of the two samples by a supervised classification algorithm, wherein:
a. the reactivity pattern comprises reactivities of IgG antibodies to ssDNA, U1 snRNP, Ro52, Collagen III, and Apo-SAA, and reactivities of IgM antibodies to Histone III-S, and the supervised classification algorithm is logistic regression (LR); or
b. the reactivity pattern comprises reactivities of IgG antibodies to ssDNA, U1 snRNP, Sm, Apo-SAA, and Ro52, and reactivities of IgM antibodies to H2a, and the supervised classification algorithm is quadratic discriminant analysis (QDA); or
c. the reactivity pattern comprises reactivities of IgG antibodies to ssDNA, U1 snRNP and Sm, and reactivities of IgM antibodies to Histone III-S, U1 snRNP, and Oligo21, and the supervised classification algorithm is linear discriminant analysis (LDA);
(iii) comparing the scores obtained for the two samples, and further comparing the score obtained for the second sample to a pre-determined threshold score, wherein the scores are calculated in the range of 0 to 1 and the pre-determined threshold score is 0.18; and
(iv) determining that the subject is amenable for treatment adjustment if there is a significant reduction of the score obtained for the second sample compared to the score obtained for the first sample, and if the score obtained for the second sample is within two standard deviations (SD) of the pre-determined threshold score.

42. The method of claim 41, wherein:
a. the reactivity pattern consists of reactivities of IgG antibodies to ssDNA, U1 snRNP, Ro52, Collagen III, and Apo-SAA, and reactivities of IgM antibodies to Histone III-S, and the supervised classification algorithm is LR; or
b. the reactivity pattern consists of reactivities of IgG antibodies to ssDNA, U1 snRNP, Sm, Apo-SAA, and Ro52, and reactivities of IgM antibodies to H2a, and the supervised classification algorithm is QDA; or
c. the reactivity pattern consists of reactivities of IgG antibodies to ssDNA, U1 snRNP, and Sm, and reactivities of IgM antibodies to Histone III-S, U1 snRNP, and Oligo21, and the supervised classification algorithm is LDA; or
d. the reactivity pattern consists of reactivities of IgG antibodies to ssDNA, Sm, DNAse I, Ro52, and U1 snRNP, and reactivities of IgM antibodies to Histone III-S, and the supervised classification algorithm is SVMs.

43. The method of claim 40, wherein the treatment is selected from the group consisting of: NSAIDs, corticosteroids, immunosuppressants, hydroxychloroquine, cyclophosphamide, immunomodulators, and TNF-α inhibitors, and the method comprises reducing the dose and/or frequency of the treatment or ceasing administration of the treatment to the subject.

44. A method for detecting resolution of systemic lupus erythematosus (SLE) in a subject having been diagnosed as having SLE, the method comprising the steps of:
(i) providing a first sample obtained from the subject at a first time point and a second sample obtained from the same subject at a second, subsequent time point, wherein the subject has been diagnosed as having SLE at least three years earlier of the second time point and is asymptomatic at the second time point;
(ii) exposing antibodies in each of the two samples to at least four antigens selected from the group consisting of: ssDNA, Sm, DNAse I, Histone III-S, Ro52, U1 snRNP, Collagen III, Apo-SAA, H2a, and Oligo21 to detect the respective reactivity patterns of the two samples to the at least four antigens;
(iii) calculating scores based on the reactivity patterns of the two samples by a supervised classification algorithm, in which the lower the score the greater is the probability that the subject is not afflicted with SLE;
(iv) comparing the scores obtained for the two samples; and
(v) determining that the subject has SLE resolution if there is a significant reduction of the score obtained for the second sample compared to the score obtained for the first sample.

45. The method of claim 44, further comprising reducing the dose and/or frequency of treatment or ceasing administration of treatment to the subject determined to have SLE resolution.

46. The method of claim 45, wherein the first time point precedes the second time point by at least ten years, or wherein the subject has been diagnosed as having SLE at least ten years earlier of the second time point.

47. The method of claim 45, wherein the subject is undergoing SLE treatment selected from the group consisting of: nonsteroidal anti-inflammatory drugs (NSAIDs), corticosteroids, immunosuppressants, hydroxychloroquine, cyclophosphamide, immunomodulators, and TNF-α inhibitors.

48. The method of claim 45, comprising the steps of:
(i) providing a first sample obtained from the subject at a first time point and a second sample obtained from the same subject at a second, subsequent time point, wherein the first time point precedes the second time point by at least ten years, and the subject is asymptomatic at the second time point;
(ii) exposing antibodies in each of the two samples to a plurality of antigens selected from the group consisting of: ssDNA, Sm, DNAse I, Histone III-S, Ro52, U1 snRNP, Collagen III, Apo-SAA, H2a, and Oligo21 to detect the respective reactivity patterns of the two samples to the plurality of antigens, and calculating scores, based on the reactivity patterns of the two samples, in which the lower the score the greater is the probability that the subject is not afflicted with SLE, using a supervised classification algorithm, wherein:
a. the reactivity pattern comprises reactivities of IgG antibodies to ssDNA, U1 snRNP, Ro52, Collagen III, and Apo-SAA, and reactivities of IgM antibodies to Histone III-S, and the supervised classification algorithm is logistic regression (LR); or
b. the reactivity pattern comprises reactivities of IgG antibodies to ssDNA, U1 snRNP, Sm, Apo-SAA, and Ro52, and reactivities of IgM antibodies to H2a, and the supervised classification algorithm is quadratic discriminant analysis (QDA); or
c. the reactivity pattern comprises reactivities of IgG antibodies to ssDNA, U1 snRNP, and Sm, and reactivities of IgM antibodies to Histone III-S, U1 snRNP, and Oligo21, and the supervised classification algorithm is linear discriminant analysis (LDA); or
d. the reactivity pattern comprises reactivities of IgG antibodies to ssDNA, Sm, DNAse I, Ro52, and U1 snRNP, and reactivities of IgM antibodies to Histone III-S, and the supervised classification algorithm is support vector machines (SVMs);
(iii) comparing the scores obtained for the two samples, and determining that the subject has SLE resolution if there is a reduction of at least 0.1 in the score obtained for the second sample compared to the score obtained for the first sample.
